(19) United States
(12) Patent Application Publication
(10) Pub. No.: US 2021/0033518 A1
(43) Pub. Date: Feb. 4, 2021

(54) DNA ORIGAMI BEADS FOR FLUORESCENCE QUANTIFICATION IN MICROFLUIDICS

(71) Applicant: Technical University of Denmark, Kgs. Lyngby (DK)

(72) Inventors: Denis Selnihhin, Aarhus N (DK); Jens Bæk Simonsen, Virum (DK); Finn Skou Pedersen, Aarhus V (DK)

(73) Assignee: Technical University of Denmark, Kgs. Lyngby (DK)

(21) Appl. No.: 16/966,159

(22) PCT Filed: Feb. 4, 2019
(86) PCT No.: PCT/EP2019/052640

§ 371 Date: Jul. 30, 2020

(30) Foreign Application Priority Data
Feb. 2, 2018 (EP) 18154868.6

(51) Int. Cl. G01N 15/10; G01N 33/58; G01N 33/543

(52) U.S. Cl. CPC G01N 15/1012; G01N 33/582; G01N 2015/1006; G01N 2800/22; G01N 33/54366

(57) ABSTRACT

A method is provided for calibrating a microfluidic system comprising, which method is based on using beads having a structure based on DNA origami, wherein said structure comprises a predetermined number of fluorophores. Furthermore, methods for determining the level of an antigen using microfluidics calibrated using fluorescent microbeads as provided herein. Also, methods are provided for determining the presence or state of a haematological disease by calibrated a microfluidics system.

### TECHNICAL FIELD

The present invention discloses beads comprising at least one structure based on DNA origami and fluorophores for use in microfluidics, such as flow cytometry.

### BACKGROUND

The commercial calibrators on the market today that are used to quantify numbers of fluorophores per particle(cell) from the flow cytometric fluorescence intensity are associated with several drawbacks: (a) The current calibrator beads are made of plastic and thus exhibit a high auto-fluorescence corresponding to about 200-2.000 fluorophores per bead. (b) The degree of fluorophore-labelling of commercial beads is associated with a large variation, (c) These calibrations are based on thousands of fluorophores per bead (including high auto-fluorescence) and thus the extrapolation into the lower range (50-2.000 fluorophores is associated with very high uncertainty).

DNA-origami-based standards for microscopy have been described in US 20140057805 A1.

### SUMMARY

The present invention provides beads comprising a structure based on DNA-origami, wherein said structure comprises a predetermined number of fluorophores for microfluidic applications, such as flow cytometry. The bead has several advantages over the prior art. One advantage is that the bead exhibits no or very little auto-fluorescence. Moreover, the number of fluorophores attached to the structure based on DNA-origami is known and can be precisely controlled so that the bead can be labelled with a distinct number of fluorophores down to as few as one fluorophore. Thus, calibration with the methods of the present disclosure allows quantification of antigens present in a sample at a very low level.

One advantage of the present invention lies in that the origami structure allows fluorescence quantification in microfluidics, where the signal is obtained differently compared to fluorescence microscopy. In microscopy, the object is stationary and thus the fluorophores attached to the object have more excitation exposure and emission time to generate the signal, while in microfluidics, such as flow cytometry, the beads are moving rapidly past the detector, which substantially limits the exposure and detection time. The calibration of flow cytometry systems may require beads with specific number of fluorophores, up to 1500-3000 fluorophores.

One aspect of the present disclosure relates to a method of calibrating a microfluidics system comprising:
a. providing at least one bead having a structure based on DNA origami, wherein said structure comprises a predetermined number of fluorophores;
b. measuring the fluorescence of said at least one bead; and
c. calibrating the microfluidics system on the basis of the fluorescence measurement of b.
   thereby obtaining a calibration curve of the microfluidics system

Calibration of a microfluidics systems, such as flow cytometry setups, using calibration beads as defined herein allows precise biochemical analysis of small biological particles e.g. extracellular vesicles, viruses and bacteria. Thus, the provided methods for calibrating microfluidics systems can advantageously be used in academic, industrial as well as for clinical analyses, including diagnostic methods based on microfluidics, such as flow cytometry.

Another aspect of the present disclosure relates to a method for determining the level of an antigen in a sample by a microfluidics system comprising
a. calibrating a microfluidics system according to the method of any one of the preceding claims, thereby obtaining a calibration curve;
b. providing a sample comprising at least one antigen, wherein said antigen is attached to a cell or to a vesicle or to a virus-like particle or a viral particle or to an extracellular vesicle;
c. contacting the sample with at least one ligand binding molecule coupled to a fluorescent label, wherein said ligand binds the at least one antigen;
d. measuring the fluorescence of said fluorescence label;
e. scoring the fluorescence of said fluorescence label based on the calibration curve, and
f. determining the level of said at least one antigen in the sample.

Another aspect of the present disclosure relates to a method for determining the presence or state of a haematological disease in an individual by a microfluidics system, such as a flow cytometry system, the method comprising
a. calibrating a microfluidics system according to the method disclosed herein, thereby obtaining a calibration curve;
b. providing a blood sample from said individual comprising at least one blood cell;
c. contacting the sample with at least one ligand binding molecule coupled to a fluorescent label, wherein said ligand is associated with the presence or state of said haematological disease;
d. measuring the fluorescence of said fluorescent label;
e. scoring the fluorescence of said fluorescent label based on the calibration curve; and
f. determining the level and/or concentration of said at least one antigen associated with the presence or state of said haematological disease.

Another aspect of the present disclosure relates to a kit for calibration of microfluidic instruments comprising a bead having a structure based on DNA origami wherein said structure comprises a predetermined number of fluorophores.

### DESCRIPTION OF DRAWINGS

FIG. 1. Example of a possible design of flow cytometry calibration bead. A: Side view of a DNA-based monomer bead. B: Front view of monomer. C: Dimerization of a whole particle. D: Dimer bead. E: TEM characterization of dimer beads. TEM images are 200*200 nm. F: Positioning of fluorophores in a dimer bead. G: Flow cytometry data and analysis of dimer beads labelled with 0, 120 and 242 Cy5 molecules by Cytoflex flow cytometry system. H: Alternative positioning of the fluorophores on outer surface of the bead.

FIG. 2. Proposition of a possible bead design based on 51 kb scaffold. A. Front view of number 8 shaped bead B. Zoom in on fluorophore strand attachment to a complementary toehold protruding from the structure.

### DETAILED DESCRIPTION

Definitions

The term “structure based on a DNA origami” as used herein refers to a DNA origami formed from a scaffold DNA strand and shorter DNA segments, also called staple strands, which form a predetermined structure of the scaffold DNA strand. The scaffold DNA strand is usually a long single-stranded DNA molecule. The length of the scaffold DNA strand depends on the system used for its production. Scaffold DNA strands of approximately 7.000 nucleotides may be produced using for example M13mp18 phage. Scaffold DNA strands of approximately 50.000 nucleotides may be produced using for example a λ/M13 hybrid virus as described in Marchi et al. (2014). The structure based on a DNA strand can comprise further components such as dyes, plasmonic structures, biological molecules such as proteins, enzymes, nanoparticles, and small molecules such as biotin. Alternatively, the structure based on a DNA origami can also be constructed solely from short DNA segments, as recently described by Ong et al. (2017). Constructing a structure based on a DNA origami solely from short DNA segments is also possible for large structures, such as structures in the gigadalton range.

The term “DNA” as used herein, is understood to mean not only strands of deoxyribonucleic acid, but also analogous structures, such as strands of ribonucleic acids (RNA), peptide nucleic acid (PNA), as well as hybrid structures.

The term “shorter DNA segments” as used herein refers to the nucleotide molecules which are also referred to as “staple strands” or “staple DNA strands” and which have a sequence complementary to a sequence of the scaffold DNA strand or another shorter DNA segment. Furthermore, said shorter DNA segments can be used to fold the long DNA strand into the predetermined structure. Alternatively, the shorter DNA segments can be those which hybridize with the DNA scaffold strand of the DNA origami in predetermined regions. The shorter DNA segments may comprise a sequence that does not hybridize to the DNA scaffold strand, called “sticky end” herein, which can be used for decorating the structure with fluorophores. The shorter DNA segments can also be used to bind/stick individual DNA origami structures together into large DNA origami oligomers in a controllable manner.

The term “calibration” is understood here to mean quantifying a measured variable on the basis of one or more reference samples or determining the properties of an apparatus, such as the sensitivity and resolution. For example, the methods disclosed herein may be used for calibration of a flow cytometer.

Structure Based on DNA Origami

In one aspect the present disclosure relates to a method of calibrating a microfluidics system, such as a flow cytometer, comprising providing at least one bead having a structure based on DNA origami, wherein said structure comprises a predetermined number of fluorophores.

The structure based on DNA origami usually comprises a single stranded DNA molecule, also called DNA scaffold strand, which is folded into a predetermined structure with the help of a plurality of staple strands. Each staple strand hybridizes to specific parts of sequence on the single stranded DNA molecule thereby determining how the single stranded DNA molecule folds.

Depending on the length of the DNA scaffold strand, a certain number of staple DNA strands will be required for folding the scaffold strand into the predesigned structure.

Thus, in one specific embodiment the structure comprises a predetermined number of fluorophores, a single stranded DNA molecule and staple DNA strands, wherein the number of staple DNA strands (Ns) is as in the formula below:

Ns=Lss/n,

wherein Lss is the length of the single stranded DNA molecule, and

wherein n is an integer number comprised between 26 and 34.

In another specific embodiment, the structure comprises a predetermined number of fluorophores, a single stranded DNA molecule and staple DNA strands, wherein the number of staple DNA strands (Ns) is as in the formula below:

Ns=Lss/n,

wherein Lss is the length of the single stranded DNA molecule, and

wherein n is an integer number comprised between 28 and 32.

However, the present invention is not limited to embodiments meeting the above equation. Indeed, it is possible to produce many different DNA origami structures, which do not follow this equation.

The length of the staple DNA strands may differ from one staple DNA to the other. The minimum length is a length that allows the staple DNA strand to be stable at room temperature.

In one embodiment, the length of the staple DNA strands is in the range between 12 and 60 nucleotides, preferably between 15 and 50 nucleotides.

Thus, in one embodiment the present disclosure relates to a method of calibrating a flow cytometer comprising providing at least one bead having a structure based on DNA origami, wherein said structure comprises a predetermined number of fluorophores, a single stranded DNA molecule and staple DNA strands.

The length of the single stranded DNA molecule is important for the size of the bead as well as for the number of fluorophores that the bead can accommodate.

Thus, the single stranded DNA molecule or DNA scaffold strand may have a length in a range between 1.500-nucleotides and 70.000-nucleotides, such as between 2.000-nucleotides and 68.000-nucleotides, such as between 5.000-nucleotides and 65.000-nucleotides, for example between 10.000-nucleotides and 60.000-nucleotides, such as between 15.000-nucleotides and 55.000-nucleotides, preferably between 20.000-nucleotides and 55.000-nucleotides, such as between 50.000 and 52.000-nucleotides. Thus, the single stranded DNA molecule or DNA scaffold strand may have a length of 51.466-nucleotides.

The single stranded DNA molecule may have a length in a range between 5.000-nucleotides and 70.000-nucleotides, for example between 7.000-nucleotides and 70.000-nucleotides, such as between 10.000-nucleotides and 70.000-nucleotides, preferably between 13.000-nucleotides and 70.000-nucleotides, such as between 15.000-nucleotides and 70.000-nucleotides, for example between 18.000-nucleotides and 70.000-nucleotides, such as between 20.000-nucleotides and 70.000-nucleotides, preferably between 23.000-nucleotides and 70.000-nucleotides, such as between 25.000-nucleotides and 70.000-nucleotides, for example between 28.000-nucleotides and 70.000-nucleotides, such as between 30.000-nucleotides and 70.000-nucleotides, preferably between 33.000-nucleotides and 70.000-nucleotides, such as between 35.000-nucleotides and 70.000-nucleotides, for example between 38.000-nucleotides and 70.000-nucleotides, such as between 40.000-nucleotides and 70.000-nucleotides, preferably between 43.000-nucleotides and 70.000-nucleotides, such as between 45.000-nucleotides and 70.000-nucleotides, for example between 48.000-nucleotides and 70.000-nucleotides, such as between 50.000-nucleotides and 70.000-nucleotides, preferably between 53.000-nucleotides and 70.000-nucleotides, such as between 55.000-nucleotides and 70.000-nucleotides, for example between 58.000-nucleotides and 70.000-nucleotides, such as between 60.000-nucleotides and 70.000-nucleotides, preferably between 63.000-nucleotides and 70.000-nucleotides, such as between 65.000-nucleotides and 70.000-nucleotides, for example between 5.000-nucleotides and 65.000-nucleotides, such as between 5.000-nucleotides and 60.000-nucleotides, preferably between 5.000-nucleotides and 55.000-nucleotides, such as between 5.000-nucleotides and 50.000-nucleotides, for example between 5.000-nucleotides and 45.000-nucleotides, such as between 5.000-nucleotides and 40.000-nucleotides, preferably between 5.000-nucleotides and 35.000-nucleotides, such as between 5.000-nucleotides and 30.000-nucleotides, for example between 5.000-nucleotides and 25.000-nucleotides, such as between 5.000-nucleotides and 20.000-nucleotides, preferably between 5.000-nucleotides and 15.000-nucleotides, such as between 5.000-nucleotides and 10.000-nucleotides, for example between 5.000-nucleotides and 7.000-nucleotides.

The single stranded DNA molecule may have a length of at least 5.000-nucleotides, for example at least 7.000-nucleotides, preferably at least 10.000-nucleotides, such as at least 15.000-nucleotides, for example at least 20.000-nucleotides, preferably at least 25.000-nucleotides, such as at least 30.000-nucleotides, for example at least 35.000-nucleotides, preferably at least 40.000-nucleotides, such as at least 45.000-nucleotides, for example at least 50.000-nucleotides.

In one embodiment the single stranded DNA molecule may be produced using a phage and has a length in a range between 5.000-nucleotides and 10.000 nucleotides.

In one embodiment the single stranded DNA molecule may be produced using a hybrid virus and has a length in a range between 5.000-nucleotides and 55.000 nucleotides.

It may be beneficial to produce long single stranded DNA molecules, such as single stranded DNA molecules having a length of at least 30.000-nucleotides as these long molecules can carry a higher number of fluorophores compared to single stranded DNA molecules having a length in the range of between 5.000 and 10.000-nucleotides, such as at the most 30.000-nucleotides.

For example, a bead comprising a single stranded DNA molecule of 7.000 to 12.000-nucleotides may accommodate 1 to 200 fluorophores; a bead comprising a single stranded DNA molecule of 40.000 to 50.000-nucleotides may accommodate 1 to 1500 fluorophores.

Another way to increase the number of fluorophores comprised in a bead is the production of dimeric, such as trimeric structures through hydrophobic interactions and hydrogen bond interactions.

Thus, in one embodiment the bead comprises a dimeric structure based on DNA origami. In another embodiment, the bead comprises a trimeric structure based on DNA origami. However, it is also possible to produce larger oligomer structures such as tetrameric, pentameric, hexameric, heptameric and/or larger multiple oligomeric structures.

The structure based on DNA origami may have any shape and it may be flat or curved. A certain geometry may be preferred over other geometries. For example a geometry that hides the fluorophores inside the structure to prevent undesired oligomerization of structures through possible hydrophobic interactions between the fluorophores may be preferred. Thus, in one embodiment the structure based on DNA origami may be a sphere, an hexagon, a barrel, a tube or a combination of these structures.

In one embodiment the structure based on DNA origami may be a sphere.

In one embodiment the structure based on DNA origami may be a barrel or a tube.

In one embodiment the structure based on DNA origami may be an hexagon.

In one embodiment, the structure based on DNA origami may be a combination of the proposed geometrical structures.

In one embodiment, the structure based on DNA origami comprises a positioning domain ,a fluorescent domain and/or triggering domain

A positioning domain is a domain where a fluorophore is placed on the structure. It can be one of the staple strands that are fully complementary to the scaffold strand or it can have a single stranded overhang sequence that is not complementary to scaffold strand. The overhang is used to attach a fluorophore labelled oligo that is complementary to the overhang.

The fluorescent domain is the domain where the fluorophores are attached. Thus, the fluorescent domain comprises the predetermined number of fluorophores. In addition, each bead may comprise more than one different type of fluorophore, for example, each bead may comprise two or more different fluorophores or fluorescent domains, such as 2, 3, 4, 5, 6, 7, 8, 9 or 10 different fluorophores or fluorescent domains. In a preferred embodiment, the beads comprises 1-4 different fluorophores or fluorescent domain.

In another embodiment, the DNA based origami structure comprises a triggering domain.

The triggering domain is a domain where a different type of fluorophore or nanoparticle can be attached. The nanoparticle e.g. gold or silver nanoparticle can efficiently spread light. Thereby, these fluorophores or nanoparticles in the triggering domain can be used to trigger detection of a calibration bead.

An advantage of using beads comprising a structure based on DNA origami is that a precise and known number of fluorophores can be attached on the structure, and therefore accurate calibration of the measuring instrument, in this case of the flow cytometer, is possible.

In one embodiment the fluorescent domain comprises sticky ends and the fluorophores are conjugated to a DNA fragment complementary to said sticky ends, so that the fluorophores are attached to said sticky ends via said complementary DNA fragment.

A bead of the present disclosure comprises a predetermined number of fluorophores. Based on the length and the design of the structure based on DNA origami, the bead may comprise a certain maximum number of fluorophores.

In one embodiment the structure based on DNA origami comprises between 1 and 50 fluorophores, such as between 1 and 80 fluorophores, such as between 1 and 100 fluorophores, such as between 1 and 150 fluorophores, such as between 1 and 200 fluorophores, such as between 1 and 300 fluorophores, such as between 1 and 400 fluorophores, such as between 1 and 500 fluorophores, such as between 1 and 600 fluorophores, such as between 1 and 700 fluorophores, such as between 1 and 800 fluorophores, such as between 1 and 900 fluorophores, preferably at the most 1000 fluorophores, such as at the most 1200 fluorophores, such as at the most 1500 fluorophores, such as at the most 1800 fluorophores, such as at the most 2000 fluorophores, such as at the most 2200 fluorophores, such as at the most 2500 fluorophores, such as at the most 2700 fluorophores, such as at the most 3000 fluorophores.

In one embodiment the bead comprises a dimeric or trimeric structure based on DNA origami, and each monomer comprises between 1 and 1800 fluorophores.

In one embodiment the bead comprises a monomeric structure based on DNA origami and said structure comprises between 1 and 900 fluorophores, such as between 1 and 1500 fluorophores, preferably between 1 and 1800 fluorophores, such as between 1 and 2700 fluorophores, preferably at the most 3000 fluorophores.

In one embodiment the bead comprises a structure based on DNA origami, wherein said structure comprises a single stranded DNA molecule of at least 30.000-nucleotides and comprising between 1 and 1000 fluorophores.

In one embodiment the bead comprises a structure based on DNA origami, wherein said structure comprises a single stranded DNA molecule of at least 50.000-nucleotides and comprising between 1 and 1800 fluorophores.

Any fluorophore suitable for flow cytometry can be used. However, the preferred fluorophores are selected from small organic molecules.

In one embodiment, the small organic molecules are smaller than 5 nm.

In one embodiment, the fluorophore is selected from a group consisting of Quasar® 566 nm (Cy 3), Quasar® 670 nm (Cy 5), and fluorescein isothiocyanate (FITC).

In one embodiment the structure based on DNA origami comprises 120 fluorophores, for example 120 Cy5 fluorescence molecules.

In one embodiment the structure based on DNA origami comprises 242 fluorophores, for example 242 Cy5 fluorescence molecules.

Suitable fluorophores are known to the skilled person and include Alexa Fluor® 488, Alexa Fluor® 532, Alexa Fluor® 647, ATTO 488 and ATTO 532 and 5/6 carboxy-tetramethyl rhodamine (TMR), 6-carboxyfluorescein (6-FAM), Alexa Fluor® 350, DY-415, ATTO 425, ATTO 465, Bodipy® FL, fluorescein isothiocyanate, Oregon Green® 488, Oregon Green® 514, Rhodamine Green™, 5′-Tetrachloro-Fluorescein, ATTO 520, 6-carboxy-4′,5′-dichloro-2′,7′-dimethoxyfluoresceine, Yakima Yellow™ dyes, Bodipy® 530/550, hexachloro-fluorescein, Alexa Fluor® 555, DY-549, Bodipy® TMR-X, cyanine phosphoramidites (cyanine 3, cyanine 3.5, cyanine 5, cyanine 5.5), ATTO 550, Rhodamine Red™, ATTO 565, Carboxy-X-Rhodamine, Texas Red (Sulforhodamine 101 acid chloride), LightCycler® Red 610, ATTO 594, DY-480-XL, DY-610, ATTO 610, LightCycler® Red 640, Bodipy 630/650, ATTO 633, Bodipy 650/665, ATTO 647N, DY-649, LightCycler® Red 670, ATTO 680, LightCycler® Red 705, DY-682, ATTO 700, ATTO 740, DY-782, IRD 700 and IRD 800, CAL Fluor® Gold 540 nm, CAL Fluor® Gold 522 nm, CAL Fluor® Gold 544 nm , CAL Fluor® Orange 560 nm, CAL Fluor® Orange 538 nm, CAL Fluor® Orange 559 nm, CAL Fluor® Red 590 nm, CAL Fluor® Red 569 nm, CAL Fluor® Red 591 nm, CAL Fluor® Red 610 nm, CAL Fluor® Red 590 nm, CAL Fluor® Red 610 nm, CAL Fluor® Red 635 nm, Quasar® 570 nm, Quasar® 548 nm, Quasar® 566 nm (Cy 3), Quasar® 670 nm, Quasar® 647 nm, Quasar® 670 nm (Cy 5), Quasar® 705 nm, Quasar® 690 nm, Quasar® 705 nm (Cy 5.5), Pulsar® 650 Dyes, SuperRox® Dyes.

In one embodiment the structure based on DNA origami comprises a DNA scaffold of a length between 50.000 and 52.000-nucleotides, for example of a length of 51.466-nucleotides; a number of staple strands between 1000 and 2000, for example between 1500 and 1600, preferably 1550; and between 50 and 1800 fluorophores.

In one embodiment the structure based on DNA origami comprises a DNA scaffold of a length between 50.000 and 52.000-nucleotides, for example of a length of 51.466-nucleotides; a number of staple strands between 1000 and 2000, for example between 1500 and 1600, preferably 1550; and between 50 and 900 fluorophores.

In one embodiment the structure based on DNA origami comprises a DNA scaffold of a length between 50.000 and 52.000-nucleotides, for example of a length of 51.466-nucleotides; a number of staple strands between 1000 and 2000, for example between 1500 and 1600, preferably 1550; and between 50 and 1800 fluorophores, for example 80 fluorophores, for example 160 fluorophores, such as 900 fluorophores, such as 1800 fluorophores.

In one embodiment the structure based on DNA origami comprises a DNA scaffold of a length between 50.000 and 52.000-nucleotides, for example of a length of 51.466-nucleotides; a number of staple strands between 1000 and 2000, for example between 1500 and 1600, preferably 1550; and 80 fluorophores, for example 80 Cy5 fluorescence molecules.

In one embodiment the structure based on DNA origami comprises a DNA scaffold of a length between 50.000 and 52.000-nucleotides, for example of a length of 51.466-nucleotides; a number of staple strands between 1000 and 2000, for example between 1500 and 1600, preferably 1550; and 160 fluorophores, for example 160 Cy5 fluorescence molecules.

Method of Calibrating a Microfluidics System

Microfluidic systems involves setups with very small volumes of fluids, and microfluidic devices utilises the principles of microfluidics and can thereby exploit the physical and chemical properties of liquids and gases at the microscale. Many operations can be executed at the same time thanks to their compact size and the shortened experiment time while they still offer an excellent data quality.

The current invention relates to methods for using DNA origami in calibration of a microfluidics system.

So, one aspect the present disclosure relates to a method of calibrating a microfluidic device comprising:
a. providing at least one bead having a structure based on DNA origami, wherein said structure comprises a predetermined number of fluorophores;
b. measuring the fluorescence of said at least one bead; and
c. calibrating the microfluidic device on the basis of the fluorescence measurement of b.
   thereby obtaining a calibration curve of the microfluidic device.

Another aspect of the present disclosure relates to a method for determining the level of an antigen in a sample by a microfluidic technology comprising
a. calibrating a microfluidic device according to the method disclosed herein, thereby obtaining a calibration curve;
b. providing a sample comprising at least one antigen, wherein said antigen is attached to a cell or to a vesicle or to a virus-like particle or a viral particle or to an extracellular vesicle;
c. contacting the sample with at least one ligand binding molecule coupled to a fluorescent label, wherein said ligand binds the at least one antigen;
d. measuring the fluorescence of said fluorescence label;
e. scoring the fluorescence of said fluorescence label based on the calibration curve, and
f. determining the level of said at least one antigen in the sample.

In a preferred embodiment, the microfluidic system is a flow cytometer system, where structures based on DNA origami, wherein said structures comprise a predetermined number of fluorophores, are used for calibration; cf. herein below. However, any specific embodiment described herein below in respect of flow cytometer systems can also be applied to microfluidic systems in general.

Method of Calibrating a Flow Cytometer

In one aspect the present disclosure relates to a method of calibrating a flow cytometer comprising:
a. providing at least one bead having a structure based on DNA origami, wherein said structure comprises a predetermined number of fluorophores;
b. measuring the fluorescence of said at least one bead; and
c. calibrating the flow cytometer on the basis of the fluorescence measurement of b.

In one embodiment the method of calibrating a flow cytometer comprises providing a series of beads each comprising a different predetermined number of fluorophores. For example at least two beads may be provided, such as at least three beads, preferably at least four beads, even more preferably at least five beads may be provided, each bead comprising a different predetermined number of fluorophores.

For example, a series of 2 beads comprising 50 and 100 fluorophores, respectively, may be provided; preferably a series of 3 beads comprising 50, 100, 200 fluorophores, respectively, may be provided; preferably a series of 4 beads comprising 50, 100, 200 and 500 fluorophores, respectively, may be provided; preferably a series of 5 beads comprising 50, 100, 200, 500 and 1000 fluorophores, respectively, may be provided; for example a series of 6 beads comprising 50, 100, 200, 500, 1000 and 1500 fluorophores, respectively, may be provided.

Thus, the fluorescence of each bead will be measured according to step b. of the method above.

Although it is possible to calibrate a flow cytometer with the methods of the present disclosure by using only two beads having a structure based on DNA origami as disclosed herein, it is preferable to use more than two beads. In fact, the precision of the calibration will increase with the number of beads used.

When the method of calibrating a flow cytometer comprises providing a series of beads each comprising a different predetermined number of fluorophores, it may be beneficial that at least one bead comprises a number of fluorophores as close as possible to the sensitivity of the method.

For example, a first bead may comprise a number of fluorophores between 1 and 50; a second bead may comprise a number of fluorophores between 51 and 100; a third bead may comprise a number of fluorophores between 101 and 150; a fourth bead may comprise a number of fluorophores between 151 and 300; a fifth bead may comprise a number of fluorophores between 301 and 500. If a series of beads comprising a number of fluorophores as in this example are used, the data for a higher number of fluorophores may easily be extrapolated.

For example, a first bead may comprise a number of fluorophores between 1 and 50; a second bead may comprise a number of fluorophores between 51 and 100; a third bead may comprise a number of fluorophores between 101 and 300; a fourth bead may comprise a number of fluorophores between 301 and 750; a fifth bead may comprise a number of fluorophores between 751 and 1500; a sixth bead may comprise a number of fluorophores between 1501 and 2700.

In one embodiment the method of calibrating a flow cytometer comprises measuring the fluorescence of the at least two beads provided, such as of the at least three beads provided, preferably of the at least four beads provided, even more preferably of the at least five beads provided.

In one embodiment the method of calibrating a flow cytometer comprises calibrating the flow cytometer on the basis of the fluorescence measurements of the at least two beads provided, such as of the at least three beads provided, preferably of the at least four beads provided, even more preferably of the at least five beads provided.

The calibration of a flow cytometer according to the methods disclosed herein allows precise determination of the sensitivity of the instrument and generation of a calibration curve. The calibration curve allow us to correlate fluorescence intensity with the number of fluorescent molecules (fluorophores) on the entity of interest for a given fluorophore.

One advantage of the calibration method of the present disclosure is that, thanks to the use of beads comprising a structure based on DNA origami and a predetermined number of fluorophores, fluorescence can be determined with precision and a linear correlation between fluorescence and number of fluorophores is determined, allowing the establishment of a zero-point. The zero-point is a reference value, which functions as a measurement of the background fluorescence e.g. the auto-fluorescence of the utilized buffer.

Thanks to this zero-point, measurements performed with different flow cytometer systems can be compared. The zero-point, together with the calibration curve, also allows precise extrapolation of values based on the fluorescence signal.

Method for Determining the Level of an Antigen

The calibration of a flow cytometer setup performed using calibration beads as described herein will allow precise biochemical analysis of small biological particles e.g. extracellular vesicles, viruses and bacteria. Such analyses can be applied in academic, industrial as well as for clinical settings

Extracellular vesicles includes exosomes and microvesicles and are secreted by almost all cells. Extracellular vesicles are generally difficult to characterize at single particle level because of their small size. Exosomes are the smallest entities with a size of 30-150 nm. These are formed by inward budding of the endosomal membrane during maturation of large multivesicular endosomes. Fusion of multivesicular endosomes with the plasma membrane results in the release of exosomes into the extracellular space. Microvesicles are larger with a size of 50-500 nm. These are released into the extracellular space by outward budding directly from the plasma membrane. Extracellular vesicle cargo is very heterogeneous and can include nucleic acids, proteins and lipids. These extracellular vesicles play an important role mediating intercellular communication. The surface proteins embedded in these exosomes can reveal their subcellular origin, producer and recipient cell type. Characterization of these vesicles at single particle level will help to understand the intercellular communication code but also allow diagnosing e.g. tumor development and progression.

One aspect of the present disclosure relates to a method for determining the level of an antigen in a sample by a microfluidic system, such as flow cytometry comprising
a. calibrating said microfluidic system, such as flow cytometer according to the method disclosed herein, thereby obtaining a calibration curve;
b. providing a sample comprising at least one antigen, wherein said antigen is attached to a cell or to a vesicle or to a virus-like particle or a viral particle or an exosome;
c. contacting the sample with at least one ligand binding molecule coupled to a fluorescent label, wherein said ligand binds the at least one antigen;
d. measuring the fluorescence of said fluorescence label;
e. scoring the fluorescence of said fluorescence label based on the calibration curve, and
f. determining the level of said at least one antigen in the sample.

In one embodiment, the method for determining the level of an antigen in a sample by flow cytometry as disclosed herein can detect as few as one single antigen in the sample provided.

In one embodiment, the sample comprises at least one antigen, such as at least 10 antigens, for example at least 25 antigens, preferably at least 50 antigens, wherein said antigen or said antigens are attached to one or more cells and/or vesicles and/or virus-like particles and/or viral particles and/or exosomes.

Method for Diagnosis of an Haematological Disease

One aspect of the present disclosure relates to a method for determining the presence or state of a haematological disease in an individual by a microfluidic system, such as flow cytometry, the method comprising
a. calibrating said microfluidic system, such as flow cytometer according to the method disclosed herein, thereby obtaining a calibration curve;
b. providing a fluid sample from said individual comprising at least one blood cell;
c. contacting the sample with at least one ligand binding molecule coupled to a fluorescent label, wherein said ligand is associated with the presence or state of said haematological disease;
d. measuring the fluorescence of said fluorescent label;
e. scoring the fluorescence of said fluorescent label based on the calibration curve; and
f. determining the level and/or concentration of said at least one antigen associated with the presence or state of said haematological disease.

In one embodiment, the fluid is selected from a group consisting of blood, bone marrow, spinal fluid, bronchoalveolar lavage and serous effusions.

In one embodiment, the individual is human.

In one embodiment the method for diagnosis of a haematological disease by flow cytometry further comprises a step of comparing said level and/or concentration of said at least one antigen on the blood cell with a cut-off value,

wherein said cut-off value is determined from the concentration range of said at least one antigen in healthy individuals, such as individuals who do not present with a haematological disease, and

wherein a level and/or concentration that is lower or greater than the cut-off value indicates the presence or absence of said haematological disease.

The fluid sample can be a blood sample, and the blood sample can be full blood, but more preferably plasma, serum, cell-free or a sample comprising at least one blood cell or blood cell derived microvesicle.

For example the methods disclosed herein can be used for diagnosis of leukemia or lymphoma by performing an immunophenotyping of a fluid sample, such as by determining the level of certain markers expressed on cells of any of the above mentioned fluid samples.

For example the following leukemias and lymphomas may be diagnosed using the methods disclosed herein: acute myelogenous leukemia (or acute myeloid leukemia), acute lymphoblastic leukemia, chronic lymphocytic or myelocytic leukemias, B-cell and T-cell non-Hodgkin lymphomas, erythroleukemia (RBC leukemia), megaloblastic leukemia (platelets), and multiple myeloma.

In one embodiment, the level of any one of the following antigens may be determined: ZAP-70, HLA-DR, TdT, CD34, CD38, CD117, CD19, CD20, CD22, CD79a, immunoglobulin heavy (gamma, alpha, mu or delta) and light chains (kappa or lambda), CD10 (pre-B cell), CD2, CD3, CD5, CD7, CD4, CD8, MPO (myeloperoxidase), CD11, CD13, CD15, CD16b, CD33, CD66, CD11 b, CD16, CD56, CD31, CD36, CD41, CD42, CD61, CD235a, CD14, CD33, CD64, CD68, CD11c, and CD103. The skilled person will know which antigens are expressed by the different types of blood cells and which levels are expected in an individual affected by a leukemia or lymphoma; see for example Morice et al. 2004; Jaffe 1987; Hanson et al. 1999; Langerak et al. 2001.

Kit for Calibration of Flow Cytometry Instruments

One aspect of the present disclosure relates to a kit for calibration of microfluidic instruments, such as flow cytometry instruments comprising at least one bead having a structure based on DNA origami wherein said structure comprises a predetermined number of fluorophores.

In one embodiment of the present disclosure, the at least one bead is as disclosed herein in the section “Structure based on DNA origami”.

In one embodiment of the present disclosure, the kit comprises at least two beads, for example at least three beads, such as at least 4 beads, preferably at least 5 beads, wherein each bead comprises a different predetermined number of fluorophores, as described in the section “Structure based on DNA origami”.

### EXAMPLES

### Example 1. Hexagonal Double Barrel

Assembly of the Calibration Beads

Calibration beads are self-assembled into designed structure a circular single stranded scaffold strand (8064 bp) and 211 shorter single-stranded staple DNA strands. The scaffold strand is bought from Tilibit nanosystems and staple strands are produced chemically by Integrated DNA technologies. Staples strands bind to scaffold strand parts of the scaffold strand making it fold into a predesigned structure as described in (Rothemund 2006).

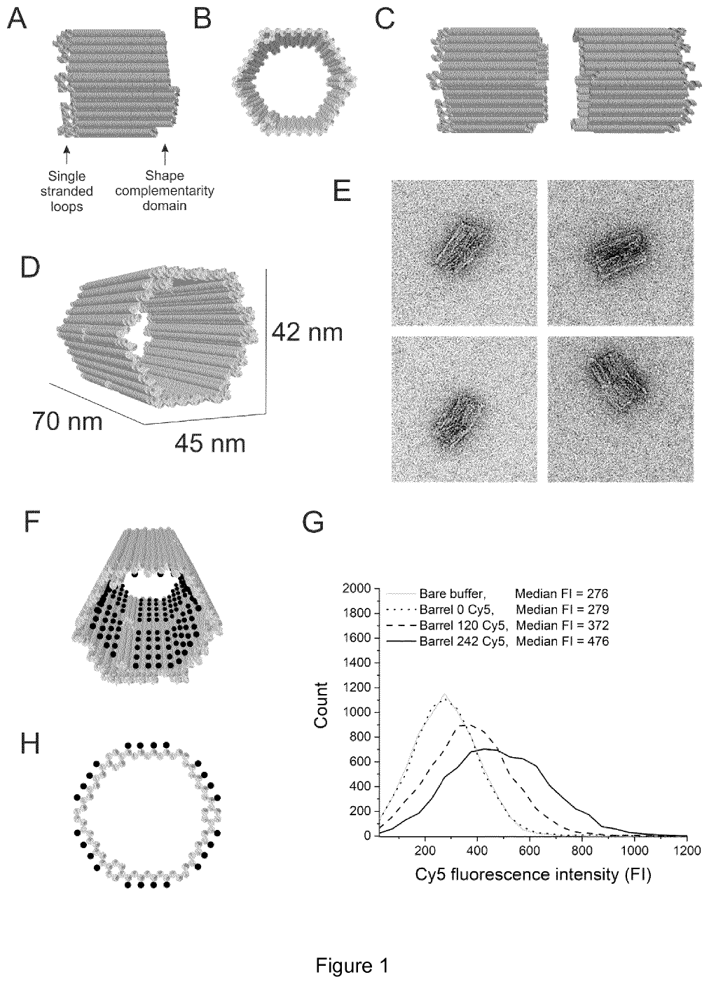

In the present case, the designed bead is assembled into a hexagonally shaped barrel (FIGS. 1A and B) that has a self-complementary side (FIG. 1A) that governs assembly of a homodimer structure (FIG. 1C) resulting in a longer hexagonal barrel with (FIGS. 1D and E). The structure has a size of approximately 42*45*70 nm. The size of transmission electron micrographs is 200*200 nm.

Some of the staple strands are labelled with fluorophores (FIG. 1F). To avoid oligomerization the structure is designed with fluorophores residing inside the barrel thus fluorophores are shielded from the environment by the structure and preventing undesired oligomerization. Analysis of beads with 0, 120 and 242 Cy5 fluorophores using Cytoflex (Beckman Coulter) flow cytometry system (FIG. 1G) shows that structures not labelled with any fluorophores do not exhibit any significant fluorescence compared with buffer alone. Addition of 120 and 242 Cy5 resulted in incremental increase in fluorescence intensity.

### Example 2. Eight-Shaped DNA Origami Structure

Assembly of the Calibration Beads

Calibration beads are self-assembled into designed structure from a circular single-stranded DNA scaffold strand (51.466 bp) and 1550 shorter single-stranded staple DNA strands. The scaffold strand is produced biologically as described in (Marchi, Saaem et al. 2014) while staple strands are produced chemically. Staple strands bind to complementary parts of the scaffold strand making it fold into a predesigned structure as described in (Rothemund 2006).

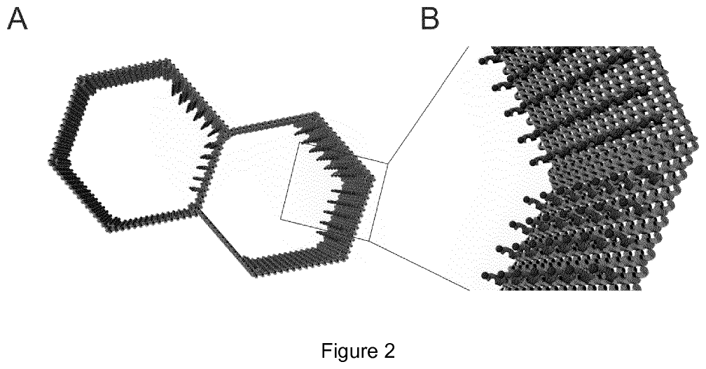

In the present case, the designed bead is assembled into two hexagonally shaped structures forming an 8 number (FIG. 2A). The structure had a size of approximately 128*220*60 nm.

Alternative way of labelling structures: some of the staple strands contain additional DNA sequences without complementarity to the scaffold strand, so called sticky ends which serve as a docking sequence for fluorophore labelled DNA strands. Hence, fluorophore labelled strands have a complementary sequence to sticky ends and are labelled with a fluorophore (FIG. 2B).

Fluorophores Attachment

Fluorophore labelled strands are produced by chemically attaching a fluorophore to a DNA strand and subsequently purifying the conjugate using high pressure liquid chromatography. The number of strands in the structure containing sticky ends determines the number of annealed fluorophore strands which equals the number of fluorophores on a bead and can range from 1 to 4000. Labelled fluorophore strands dock onto complementary sticky ends that point into the structure (FIG. 2B). This way the structure screens the fluorophores from the environment and prevents undesired oligomerization of several beads through hydrophobic fluorophore interactions, which would lead to oligomers with non-defined number of fluorophores. Alternatively, instead of sticky ends the strands in the structure can be conjugated directly to a fluorophore of interest.

Assembly of the Structure.

Design and assembly of DNA origami calibration beads. CadNano software (http://cadnano.org/) was used for generation of staple strand sequences AutoCAD Maya (http://www.autodesk.com/) was used for evaluation of the 3D shape and designing shape complementary domain for dimerization into fully assembled bead. The self-assembly of DNA origami structures was performed by mixing Single-stranded Scaffold DNA (p8064, tilibit nanosystems) with 10× excess of staple strands in 100 μL TAE/Mg2+ buffer (40 mM Trisacetate, 1 mM EDTA, pH=8.3, 12 mM Mg2+). Annealing of staple strands was done using a following ramp: 65° C. for 5 min, followed by a temperature rapid decrease to 50° C. and slow cooling for 200min for each 1° C. from 50-43° C. then rapid cooling to 20° C. In structures assembled with fluorophores, fluorophore strands were conjugated with a fluorophore in 5′end. Assembled structures were purified from excess of staple strands gel extraction. Purified structures were characterized by transmission electron microscopy and used directly for flow cytometry analysis.

Results

A Cytoflex (Beckman Coulter) cytometry system was used to test calibration beads assembled according to the description of example 1 above and labelled with none, 120 and 242 Cy5 fluorescence molecules. Cytoflex system showed a significant difference between the obtained fluorescence signal coming from unlabelled, labelled with 120 Cy5 fluorescence molecules and labelled with 242 Cy5 fluorescence molecules, respectively. Remarkably, the unlabeled calibration beads do not have any autofluorescence and the obtained fluorescence signal is equal to the signal of buffer. This allows to define a “0” point as a real point without any need for down extrapolation of the dataset. This allows the making of a calibration curve for flow cytometry systems that enables a precise quantification of flow cytometry output signals of low abundant antigens.

Bigger beads compared to the one of Example 1 can be assembled either by using a longer scaffold strand as described in Example 2 or by organizing the beads in dimers or trimers through hydrophobic interactions. This allows each bead to accommodate a larger number of fluorophores, such as up to 1500, 1800, 2000, 2500, 2700 fluorophores, and so a more diverse pool of beads to choose from for the calibration.

The quality of the synthesized beads is evaluated by cryo-TEM and agarose gel.

### BIBLIOGRAPHY

Hanson C A: Acute leukemias and myelodysplastic syndromes. In Clinical Laboratory Medicine. Edited by K D McClatchey. Baltimore, Md., Williams and Wilkins, 1994, pp 939-969.

Jaffe E S, Cossman J: Immunodiagnosis of lymphoid and mononuclear phagocytic neoplasms. In Manual of Clinical Immunology. Third edition. Edited by N R Rose, H Friedman, J L Fahey. ASM Press. 1987, pp 779-790.

Langerak, van Den Beemd, Wolvers-Tettero, et al: Molecular and flow cytometric analysis of the Vbeta repertoire for clonality assessment in mature TCR alpha beta T-cell proliferations. Blood 2001;98(1):165-173.

Marchi, A. N., I. Saaem, B. N. Vogen, S. Brown and T. H. LaBean (2014). “Toward larger DNA origami.” Nano Lett 14(10): 5740-5747.

Morice W G, Kimlinger T, Katzmann J A, et al: Flow cytometric assessment of TCR-V-beta expression in the evaluation of peripheral blood involvement by T-cell lymphoproliferative disorders: a comparison with conventional T-cell immunophenotyping and molecular genetic techniques. Am J Clin Pathol 2004; 121(3):373-383.

Ong, L. L., et al. 2017, Programmable self-assembly of three-dimensional nanostructures from 10,000 unique components. Nature 552: p. 72.

Rothemund, P. W. (2006). Folding DNA to create nanoscale shapes and patterns. Nature 440(7082): 297-302.

Tables

TABLE 1
An example of a DNA origami scaffold sequence (SEQ ID NO: 1)

GGCAATGACCTGATAGCCTTTGTAGATCTCTCAAAAATAGCTACCCTCTCCGGCATTAATTTATC
AGCTAGAACGGTTGAATATCATATTGATGGTGATTTGACTGTCTCCGGCCTTTCTCACCCTTTTG
AATCTTTACCTACACATTACTCAGGCATTGCATTTAAAATATATGAGGGTTCTAAAAATTTTTATCC
TTGCGTTGAAATAAAGGCTTCTCCCGCAAAAGTATTACAGGGTCATAATGTTTTTGGTACAACCG
ATTTAGCTTTATGCTCTGAGGCTTTATTGCTTAATTTTGCTAATTCTTTGCCTTGCCTGTATGATTT
ATTGGATGTTAATGCTACTACTATTAGTAGAATTGATGCCACCTTTTCAGCTCGCGCCCCAAATG
AAAATATAGCTAAACAGGTTATTGACCATTTGCGAAATGTATCTAATGGTCAAACTAAATCTACTC
GTTCGCAGAATTGGGAATCAACTGTTATATGGAATGAAACTTCCAGACACCGTACTTTAGTTGCA
TATTTAAAACATGTTGAGCTACAGCATTATATTCAGCAATTAAGCTCTAAGCCATCCGCAAAAATG
ACCTCTTATCAAAAGGAGCAATTAAAGGTACTCTCTAATCCTGACCTGTTGGAGTTTGCTTCCGG
TCTGGTTCGCTTTGAAGCTCGAATTAAAACGCGATATTTGAAGTCTTTCGGGCTTCCTCTTAATC
TTTTTGATGCAATCCGCTTTGCTTCTGACTATAATAGTCAGGGTAAAGACCTGATTTTTGATTTAT
GGTCATTCTCGTTTTCTGAACTGTTTAAAGCATTTGAGGGGGATTCAATGAATATTTATGACGATT
CCGCAGTATTGGACGCTATCCAGTCTAAACATTTTACTATTACCCCCTCTGGCAAAACTTCTTTT
GCAAAAGCCTCTCGCTATTTTGGTTTTTATCGTCGTCTGGTAAACGAGGGTTATGATAGTGTTGC
TCTTACTATGCCTCGTAATTCCTTTTGGCGTTATGTATCTGCATTAGTTGAATGTGGTATTCCTAA
ATCTCAACTGATGAATCTTTCTACCTGTAATAATGTTGTTCCGTTAGTTCGTTTTATTAACGTAGA
TTTTTCTTCCCAACGTCCTGACTGGTATAATGAGCCAGTTCTTAAAATCGCATAAGGTAATTCACA
ATGATTAAAGTTGAAATTAAACCATCTCAAGCCCAATTTACTACTCGTTCTGGTGTTTCTCGTCAG
GGCAAGCCTTATTCACTGAATGAGCAGCTTTGTTACGTTGATTTGGGTAATGAATATCCGGTTCT
TGTCAAGATTACTCTTGATGAAGGTCAGCCAGCCTATGCGCCTGGTCTGTACACCGTTCATCTG
TCCTCTTTCAAAGTTGGTCAGTTCGGTTCCCTTATGATTGACCGTCTGCGCCTCGTTCCGGCTAA
GTAACATGGAGCAGGTCGCGGATTTCGACACAATTTATCAGGCGATGATACAAATCTCCGTTGT
ACTTTGTTTCGCGCTTGGTATAATCGCTGGGGGTCAAAGATGAGTGTTTTAGTGTATTCTTTTGC
CTCTTTCGTTTTAGGTTGGTGCCTTCGTAGTGGCATTACGTATTTTACCCGTTTAATGGAAACTTC
CTCATGAAAAAGTCTTTAGTCCTCAAAGCCTCTGTAGCCGTTGCTACCCTCGTTCCGATGCTGTC
TTTCGCTGCTGAGGGTGACGATCCCGCAAAAGCGGCCTTTAACTCCCTGCAAGCCTCAGCGAC
CGAATATATCGGTTATGCGTGGGCGATGGTTGTTGTCATTGTCGGCGCAACTATCGGTATCAAG
CTGTTTAAGAAATTCACCTCGAAAGCAAGCTGATAAACCGATACAATTAAAGGCTCCTTTTGGAG
CCTTTTTTTTGGAGATTTTCAACGTGAAAAAATTATTATTCGCAATTCCTTTAGTTGTTCCTTTCTA
TTCTCACTCCGCTGAAACTGTTGAAAGTTGTTTAGCAAAATCCCATACAGAAAATTCATTTACTAA
CGTCTGGAAAGACGACAAAACTTTAGATCGTTACGCTAACTATGAGGGCTGTCTGTGGAATGCT
ACAGGCGTTGTAGTTTGTACTGGTGACGAAACTCAGTGTTACGGTACATGGGTTCCTATTGGGC
TTGCTATCCCTGAAAATGAGGGTGGTGGCTCTGAGGGTGGCGGTTCTGAGGGTGGCGGTTCTG
AGGGTGGCGGTACTAAACCTCCTGAGTACGGTGATACACCTATTCCGGGCTATACTTATATCAA
CCCTCTCGACGGCACTTATCCGCCTGGTACTGAGCAAAACCCCGCTAATCCTAATCCTTCTCTT
GAGGAGTCTCAGCCTCTTAATACTTTCATGTTTCAGAATAATAGGTTCCGAAATAGGCAGGGGG
CATTAACTGTTTATACGGGCACTGTTACTCAAGGCACTGACCCCGTTAAAACTTATTACCAGTAC
ACTCCTGTATCATCAAAAGCCATGTATGACGCTTACTGGAACGGTAAATTCAGAGACTGCGCTTT
CCATTCTGGCTTTAATGAGGATTTATTTGTTTGTGAATATCAAGGCCAATCGTCTGACCTGCCTC
AACCTCCTGTCAATGCTGGCGGCGGCTCTGGTGGTGGTTCTGGTGGCGGCTCTGAGGGTGGT
GGCTCTGAGGGTGGCGGTTCTGAGGGTGGCGGCTCTGAGGGAGGCGGTTCCGGTGGTGGCT
CTGGTTCCGGTGATTTTGATTATGAAAAGATGGCAAACGCTAATAAGGGGGCTATGACCGAAAA
TGCCGATGAAAACGCGCTACAGTCTGACGCTAAAGGCAAACTTGATTCTGTCGCTACTGATTAC
GGTGCTGCTATCGATGGTTTCATTGGTGACGTTTCCGGCCTTGCTAATGGTAATGGTGCTACTG
GTGATTTTGCTGGCTCTAATTCCCAAATGGCTCAAGTCGGTGACGGTGATAATTCACCTTTAATG
AATAATTTCCGTCAATATTTACCTTCCCTCCCTCAATCGGTTGAATGTCGCCCTTTTGTCTTTGGC
GCTGGTAAACCATATGAATTTTCTATTGATTGTGACAAAATAAACTTATTCCGTGGTGTCTTTGCG
TTTCTTTTATATGTTGCCACCTTTATGTATGTATTTTCTACGTTTGCTAACATACTGCGTAATAAGG
AGTCTTAATCATGCCAGTTCTTTTGGGTATTCCGTTATTATTGCGTTTCCTCGGTTTCCTTCTGGT
AACTTTGTTCGGCTATCTGCTTACTTTTCTTAAAAAGGGCTTCGGTAAGATAGCTATTGCTATTTC
ATTGTTTCTTGCTCTTATTATTGGGCTTAACTCAATTCTTGTGGGTTATCTCTCTGATATTAGCGC
TCAATTACCCTCTGACTTTGTTCAGGGTGTTCAGTTAATTCTCCCGTCTAATGCGCTTCCCTGTTT
TTATGTTATTCTCTCTGTAAAGGCTGCTATTTTCATTTTTGACGTTAAACAAAAAATCGTTTCTTAT
TTGGATTGGGATAAATAATATGGCTGTTTATTTTGTAACTGGCAAATTAGGCTCTGGAAAGACGC
TCGTTAGCGTTGGTAAGATTCAGGATAAAATTGTAGCTGGGTGCAAAATAGCAACTAATCTTGAT
TTAAGGCTTCAAAACCTCCCGCAAGTCGGGAGGTTCGCTAAAACGCCTCGCGTTCTTAGAATAC
CGGATAAGCCTTCTATATCTGATTTGCTTGCTATTGGGCGCGGTAATGATTCCTACGATGAAAAT
AAAAACGGCTTGCTTGTTCTCGATGAGTGCGGTACTTGGTTTAATACCCGTTCTTGGAATGATAA
GGAAAGACAGCCGATTATTGATTGGTTTCTACATGCTCGTAAATTAGGATGGGATATTATTTTTCT
TGTTCAGGACTTATCTATTGTTGATAAACAGGCGCGTTCTGCATTAGCTGAACATGTTGTTTATT
GTCGTCGTCTGGACAGAATTACTTTACCTTTTGTCGGTACTTTATATTCTCTTATTACTGGCTCGA
AAATGCCTCTGCCTAAATTACATGTTGGCGTTGTTAAATATGGCGATTCTCAATTAAGCCCTACT
GTTGAGCGTTGGCTTTATACTGGTAAGAATTTGTATAACGCATATGATACTAAACAGGCTTTTTCT
AGTAATTATGATTCCGGTGTTTATTCTTATTTAACGCCTTATTTATCACACGGTCGGTATTTCAAA
CCATTAAATTTAGGTCAGAAGATGAAATTAACTAAAATATATTTGAAAAAGTTTTCTCGCGTTCTTT
GTCTTGCGATTGGATTTGCATCAGCATTTACATATAGTTATATAACCCAACCTAAGCCGGAGGTT
AAAAAGGTAGTCTCTCAGACCTATGATTTTGATAAATTCACTATTGACTCTTCTCAGCGTCTTAAT
CTAAGCTATCGCTATGTTTTCAAGGATTCTAAGGGAAAATTAATTAATAGCGACGATTTACAGAA
GCAAGGTTATTCACTCACATATATTGATTTATGTACTGTTTCCATTAAAAAAGGTAATTCAAATGA
AATTGTTAAATGTAATTAATTTTGTTTTCTTGATGTTTGTTTCATCATCTTCTTTTGCTCAGGTAATT
GAAATGAATAATTCGCCTCTGCGCGATTTTGTAACTTGGTATTCAAAGCAATCAGGCGAATCCGT
TATTGTTTCTCCCGATGTAAAAGGTACTGTTACTGTATATTCATCTGACGTTAAACCTGAAAATCT
ACGCAATTTCTTTATTTCTGTTTTACGTGCAAATAATTTTGATATGGTAGGTTCTAACCCTTCCATT
ATTCAGAAGTATAATCCAAACAATCAGGATTATATTGATGAATTGCCATCATCTGATAATCAGGAA
TATGATGATAATTCCGCTCCTTCTGGTGGTTTCTTTGTTCCGCAAAATGATAATGTTACTCAAACT
TTTAAAATTAATAACGTTCGGGCAAAGGATTTAATACGAGTTGTCGAATTGTTTGTAAAGTCTAAT
ACTTCTAAATCCTCAAATGTATTATCTATTGACGGCTCTAATCTATTAGTTGTTAGTGCTCCTAAA
GATATTTTAGATAACCTTCCTCAATTCCTTTCAACTGTTGATTTGCCAACTGACCAGATATTGATT
GAGGGTTTGATATTTGAGGTTCAGCAAGGTGATGCTTTAGATTTTTCATTTGCTGCTGGCTCTCA
GCGTGGCACTGTTGCAGGCGGTGTTAATACTGACCGCCTCACCTCTGTTTTATCTTCTGCTGGT
GGTTCGTTCGGTATTTTTAATGGCGATGTTTTAGGGCTATCAGTTCGCGCATTAAAGACTAATAG
CCATTCAAAAATATTGTCTGTGCCACGTATTCTTACGCTTTCAGGTCAGAAGGGTTCTATCTCTG
TTGGCCAGAATGTCCCTTTTATTACTGGTCGTGTGACTGGTGAATCTGCCAATGTAAATAATCCA
TTTCAGACGATTGAGCGTCAAAATGTAGGTATTTCCATGAGCGTTTTTCCTGTTGCAATGGCTGG
CGGTAATATTGTTCTGGATATTACCAGCAAGGCCGATAGTTTGAGTTCTTCTACTCAGGCAAGTG
ATGTTATTACTAATCAAAGAAGTATTGCTACAACGGTTAATTTGCGTGATGGACAGACTCTTTTAC
TCGGTGGCCTCACTGATTATAAAAACACTTCTCAGGATTCTGGCGTACCGTTCCTGTCTAAAATC
CCTTTAATCGGCCTCCTGTTTAGCTCCCGCTCTGATTCTAACGAGGAAAGCACGTTATACGTGCT
CGTCAAAGCAACCATAGTACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTTGGGTGATGGTTCACGTAGT
GGGCCATCGCCCTGATAGACGGTTTTTCGCCCTTTGACGTTGGAGTCCACGTTCTTTAATAGTG
GACTCTTGTTCCAAACTGGAACAACACTCAACCCTATCTCGGGCTATTCTTTTGATTTATAAGGG
ATTTTGCCGATTTCGGAACCACCATCAAACAGGATTTTCGCCTGCTGGGGCAAACCAGCGTGGA
CCGCTTGCTGCAACTCTCTCAGGGCCAGGCGGTGAAGGGCAATCAGCTGTTGCCCGTCTCACT
GGTGAAAAGAAAAACCACCCTGGCGCCCAATACGCAAACCGCCTCTCCCCGCGCGTTGGCCGA
TTCATTAATGCAGCTGGCACGACAGGTTTCCCGACTGGAAAGCGGGCAGTGAGCGCAACGCAA
TTAATGTGAGTTAGCTCACTCATTAGGCACCCCAGGCTTTACACTTTATGCTTCCGGCTCGTATG
TTGTGTGGAATTGTGAGCGGATAACAATTTCACACAGGAAACAGCTATGACCATGATTACGAATT
CGAGCTCGGTACCCGGGGATCCTCAACTGTGAGGAGGCTCACGGACGCGAAGAACAGGCACG
CGTGCTGGCAGAAACCCCCGGTATGACCGTGAAAACGGCCCGCCGCATTCTGGCCGCAGCAC
CACAGAGTGCACAGGCGCGCAGTGACACTGCGCTGGATCGTCTGATGCAGGGGGCACCGGCA
CCGCTGGCTGCAGGTAACCCGGCATCTGATGCCGTTAACGATTTGCTGAACACACCAGTGTAA
GGGATGTTTATGACGAGCAAAGAAACCTTTACCCATTACCAGCCGCAGGGCAACAGTGACCCG
GCTCATACCGCAACCGCGCCCGGCGGATTGAGTGCGAAAGCGCCTGCAATGACCCCGCTGAT
GCTGGACACCTCCAGCCGTAAGCTGGTTGCGTGGGATGGCACCACCGACGGTGCTGCCGTTG
GCATTCTTGCGGTTGCTGCTGACCAGACCAGCACCACGCTGACGTTCTACAAGTCCGGCACGT
TCCGTTATGAGGATGTGCTCTGGCCGGAGGCTGCCAGCGACGAGACGAAAAAACGGACCGCG
TTTGCCGGAACGGCAATCAGCATCGTTTAACTTTACCCTTCATCACTAAAGGCCGCCTGTGCGG
CTTTTTTTACGGGATTTTTTTATGTCGATGTACACAACCGCCCAACTGCTGGCGGCAAATGAGCA
GAAATTTAAGTTTGATCCGCTGTTTCTGCGTCTCTTTTTCCGTGAGAGCTATCCCTTCACCACGG
AGAAAGTCTATCTCTCACAAATTCCGGGACTGGTAAACATGGCGCTGTACGTTTCGCCGATTGT
TTCCGGTGAGGTTATCCGTTCCCGTGGCGGCTCCACCTCTGAAAGCTTGGCACTGGCCGTCGT
TTTACAACGTCGTGACTGGGAAAACCCTGGCGTTACCCAACTTAATCGCCTTGCAGCACATCCC
CCTTTCGCCAGCTGGCGTAATAGCGAAGAGGCCCGCACCGATCGCCCTTCCCAACAGTTGCGC
AGCCTGAATGGCGAATGGCGCTTTGCCTGGTTTCCGGCACCAGAAGCGGTGCCGGAAAGCTG
GCTGGAGTGCGATCTTCCTGAGGCCGATACTGTCGTCGTCCCCTCAAACTGGCAGATGCACGG
TTACGATGCGCCCATCTACACCAACGTGACCTATCCCATTACGGTCAATCCGCCGTTTGTTCCC
ACGGAGAATCCGACGGGTTGTTACTCGCTCACATTTAATGTTGATGAAAGCTGGCTACAGGAAG
GCCAGACGCGAATTATTTTTGATGGCGTTCCTATTGGTTAAAAAATGAGCTGATTTAACAAAAATT
TAATGCGAATTTTAACAAAATATTAACGTTTACAATTTAAATATTTGCTTATACAATCTTCCTGTTTT
TGGGGCTTTTCTGATTATCAACCGGGGTACATATGATTGACATGCTAGTTTTACGATTACCGTTC
ATCGATTCTCTTGTTTGCTCCAGACTCTCA

TABLE 2
Examples of staple strand sequences suitable for
hexagonal barrel structure

| | SEQ ID NO |
|---|---|
| Flurophore strand sequence | |
| ATACAGGAATGCCTGAGTAATATCAATA | 2 |
| CTCAGAGGCAAGGATAAAAATGGAGAGG | 3 |
| TTCCCAAGAGTAGATTTAGTTCTAAATC | 4 |
| CTGAATAAAAAGGTGGCATCATAGTAGT | 5 |
| TAAAGTACAATAACCTGTTTAAAGAATT | 6 |
| AAATCAGATTGCTCCTTTTGAGTAGCTC | 7 |
| GGATTGCCGGAAGCAAACTCCTGGAAGT | 8 |
| GGCGAAGTTTTGCCAGAAGATTAA | 9 |
| GTGTCCCCCTCAAATGCTCAGAAA | 10 |
| ACCATAGCGTCCAATACCCCTGAC | 11 |
| GGTTGTAGGGAGAAGCCTTTAGAGATCT | 12 |
| AGCATTATAAAGATTCAAAAGGCCGGAG | 13 |
| AGCAAAAACCCTCATATATTTTCTAGCT | 14 |
| AACATGTTTTCATTTGGGGCGATAAATC | 15 |
| TTCATTCTAGATACATTTCGCATAAAGC | 16 |
| GAGGAAGGAGCTTCAAAGCGACAGTTGA | 17 |
| ACGAGAATCATTTTTGCGGATTTAATTG | 18 |
| TATTATACAGGATTAGAGAGTATGCAAC | 19 |
| ATAATAGTAAAATGTTTGCAAAGC | 20 |
| CCCTGACGGTTCGTCATAAATATTAAATCAA | 21 |
| ACATAACTTGGGAAGGCAAAAAGGAAGTTTCCATTGCATAAC | 22 |
| GCGATTATACGCAACGGCTACAGAAAGGCCG | 23 |
| CAACCATATTTCTTAAACAGCGTTAGTA | 24 |
| CAGCAGCTTTTTTCACGTTGAAGGAATT | 25 |
| CTTGCAGAGCCTTTAATTGTATTCAACA | 26 |
| AAAGGAACACTGAGTTTCGTCTCAGAGC | 27 |
| TTTGCTATTCCACAGACAGCCGTACCGC | 28 |
| CGAGGCACTCATTAACTCATCAGGACTAAAGACTTGCTGAGG | 29 |
| CAATAAAATACGTAATGATGACAA | 30 |
| AGTACAAGACAGCATCGGAACTCACCCT | 31 |
| CTTTTGCCAAAAAAAAGGCTCAGAATAG | 32 |
| CGATATAATCAGCTTGCTTTCATGGGAT | 33 |
| AATGAATTTAGCGTAACGATCTCACCGT | 34 |
| GCGAATAATAGGAACCCATGTTCAGGGA | 35 |
| GTTTCAGCAAACTACAACGCCCCACCCT | 36 |
| ATTATTCTAGCGACAGAATCATAGCCCC | 37 |
| CATTTGGAAACGTCACCAATGCACCGGA | 38 |
| TAGCAAAATAAAAGAAACGCAAGCCAGC | 39 |
| ACCAGAACAAAAGGGCGACATGAGGGAG | 40 |
| AAAAGAACACAATCAATAGAAGTGAATT | 41 |
| TACAGAGTATCTTACCGAAGCGAGGAAA | 42 |
| GAGAATTAAGCCCAATAATAAGATTAAG | 43 |
| AATCCAAATAAGAAACGTAACGTC | 44 |
| TTACTGAATCTTACCAACACCCTG | 45 |
| AACTTTGCCAGTTACAACATAAAA | 46 |
| AAAATCACATTACCATTAGCACGGAACC | 47 |
| GGAAGGTCTTTAGCGTCAGACTTTCATCG | 48 |
| ATCACCGCGATAGCAGCACCGCCATCTT | 49 |
| CGCAATAATGGTTTACCAGCGGACGGAA | 50 |
| ACTCCTTCACGGAATAAGTTTCTTGAGC | 51 |
| AACAAAGATAACCCACAAGAAAGTATGT | 52 |
| AAAAATGAAGAAAAGTAAGCACAAAGTT | 53 |
| ACAGGGAAAACAATGAAATAGAATACCC | 54 |
| AGAAGCCATATTATTTACAGCCTT | 55 |
| TTTGAGCGTCTTTCCAGTAGACGG | 56 |
| AGATATAATTTTCATGTTTAGAAATTTAATGGTTTTAGGTCT | 57 |
| ACTCATCAGAACGCGAGGCATAATCGGCAAACCAAAGCC | 58 |
| AACAACTTTTTCAAATAGTTATAT | 59 |
| ATTTATCCATAGCGATAGCTTTTCAATT | 60 |
| CAAATCCTACATAAATCAATACTTTTTT | 61 |
| CCGGCTTAATCGTCGCTATTAGAAAACA | 62 |
| TTCATTTTATACAGTAACAGTTTTGCAC | 63 |
| GAAACAAGATTCGCCTGATTGTTCTGAA | 64 |
| AATGGAATCAGGTTTAACGTCAAGAAAT | 65 |
| GAGAGACTTCCCTTAGAATCCAGATGAT | 66 |
| TATTCTAGAGAACATACAAATGTTAATTTCATCTTTTAACCT | 67 |
| AAATTAAACATCGGGAGAAACGAACCTA | 68 |
| CTTAATTGCAAGACAAAGAACGCTGATG | 69 |
| AACTATAGTGAATAACCTTGCTAACAAT | 70 |
| ACCGGAATCATAATCGACCGTGTGATAAATAGTGA | 71 |
| ACCTGAGTACCAAGTTACAAATCCTGAT | 72 |
| AGAACTCGACATTCTGGCCAATATTTTT | 73 |
| ATTACCGTACATTGGCAGATTCCCTAAA | 74 |
| TAAAGGGGGAACGGTACGCCATTGCAAC | 75 |
| GGCGCGTCCGTTGTAGCAATATAGTAAT | 76 |
| TTCCTCGTGAGGCCACCGAGTCGGCCTT | 77 |
| ATCACCCGGTCACGCTGCGCGTTGCTTT | 78 |
| ACTAAATGAAAGCGAAAGGAGCAGAGCG | 79 |
| AATCGGCATAAATCAAAAGAACTAAAGG | 80 |
| ACAACGTCAAAGGGCGAATCAGGG | 81 |
| CCCCAGTTTGGAACAAGGTTTTTT | 82 |
| AGGAAAAAATCGTCTGAAATGAATACCG | 83 |
| AACATCAAGAACCCTTCTGACAAGAATA | 84 |
| GCTGGTACACACGACCAGTAACTTTAAT | 85 |
| GACGAGCTCTGTCCATCACGCGAGTAGA | 86 |
| GGAGCTAGAGAAGTGTTTTTAGAACAAT | 87 |
| GAGCCCCCGTGGCGAGAAAGGGGCCGAT | 88 |
| CGATGGCCACACCCGCCGCGCGCTACAG | 89 |
| GGGGTCGTAGGGCGCTGGCAAACGTGCT | 90 |
| CATTATTAAAGAACGTGGTGAACC | 91 |
| TTTAGATAGGGTTGAGTGTAAAGC | 92 |
| GCTAACTTCCAGTCAATCGTTTCGGTGGTGCCATCAACGTCA | 93 |
| TAGCTCTGCCGCCACGGGAACAGGGCGA | 94 |
| TGCCGGACCGCACAGGCGGCCAAAGAGA | 95 |
| CGCTGGCTGCTGATTGCCGTTGAGATAG | 96 |
| TTAAATTACGTTGTAAAACGAGAAAGGG | 97 |
| CAGCAACGGCGGTTGTGTACAATTTGCC | 98 |
| GTTGCAGGCCAACGCTTTGCTTCCAGCATCAGCGGGGCCAGA | 99 |
| TCACCGCGTTTGCGGCTGGTAGCAGGCGCTTTCGCGTCTCGT | 100 |
| GGGGCCAGCTCTTACACACCAGCTTACGGCTCGGAACG | 101 |
| CCGGGTTAGAATGCCAACGGCTGGTCAG | 102 |
| GCGTGGTAAAAAAATCCCGTAATCAAAC | 103 |
| GCACATCATGAAGGGTAAAGTGAAGGGA | 104 |
| ACTTTCTCTCACCGGAAACAACAGGCTG | 105 |
| GCCAGCAGCCAGGGTTTTCCCGCGATTA | 106 |
| CGCAGAATGCCAAGCTTTCAGTTCGCTA | 107 |
| ACTCAGGTAGCCCGGAATAGGACGGGGTCAGTGCCCTTTTGAT | 108 |
| TAGCAAGGACTCCTCAAGAGAAAAGTATTAAGAGGATTCACA | 109 |
| CACCACCGGATTAGCGGGGTTAACCTATTATTCTGCATTAAA | 110 |
| CAGAACCGTACCAGGCGGATAAATGCCCCCTGCCTCGCAGTC | 111 |
| CACCCTCTCGAGAGGGTTGATACAGTGCCCGTATATCCAGTA | 112 |
| TGTTTGGGGCAATTCATCAATCCGTCAATAGATAAAAATATC | 113 |
| TGCGTAGGTTTGAGTAACATTACGTTATTAATTTTGCTGAAC | 114 |
| GTAAAACTGCGGAACAAAGAACGTATTAAATCCTTCCTCAAT | 115 |
| CCATATCGAAGGAGCGGAATTTTTACAAACAATTCTTGGCAA | 116 |
| TAATGGATATTCCTGATTATCGAGGATTTAGAAGTGGAATTG | 117 |
| GGATGTGGAGGGGACGACGACGTAATGG | 118 |
| TTACGCCGCCTCAGGAAGATCGGAACAA | 119 |
| TCGGTGCAGCCAGCTTTCCGGCAACCCG | 120 |
| AGTTGGGTAACCGTGCATCTGGTGTAGA | 121 |
| CGCAACTTCTGGTGCCGGAAAACATTAA | 122 |
| Dimer strands | |
| CAACTTTAATCATTGTGAATTACCGTAAATTTTCA | 123 |
| AGATTTGTATCATCGGAAACAA | 124 |
| GTACAAGCGCCCTGATAATTACCCAAATCAACGTAA | 125 |
| GCCATATTTAAAGTAGGGCAAGAACGGGTATTTGTCTTTATGT | 126 |
| AACCAACGCCGAAAAATAATATCCTACGAGCCCTTATCA | 127 |
| ATCCGCTCACAAATTGCGTTG | 128 |
| CTGCAGCCAGCGGTGCTCAGATGCTCACTGCCCGCTTCACATTATTCC | 129 |
| GTCAGCACCGAACGGCACGGTGCCTTTCCTGTGTGAAATTGTT | 130 |
| CAAAGCTGCTCAGGGCTTGAGATGGTTTA | 131 |
| Structural | |
| TGATTTCAACCATAAAGTGACCATCATATAAACCA | 132 |
| ACAAAGGAAACAAGAGAATCG | 133 |
| ACAGTCAACAGGAAGATTGTATTTAAATCACGTTGCCAG | 134 |
| TGATATTATAATCAGAAAAGCTTTTGTTATTGACCAGTATCGAGCT | 135 |
| CGTTAAATGCCAAGGCAGCTATATTTTAAATACCT | 136 |
| GATAAATCAATCATATGTACCATTTTTGCTCCGTGGCAC | 137 |
| GTAGCTAGTAATCGTAAAACTTTTTTAACGAGTAACACCGCTGTTG | 138 |
| AGATGAAAAATAGCAGGAATACCACATTAATAAAAGCAC | 139 |
| AAGGGAACCGTACGAAGCGAACTAACGGAACGAGATTTGAGA | 140 |
| AATCCCAGCGATTTTAAGAACTGGTAAGAAACACCAGAACGAGTATTAT | 141 |
| CTCCATGTTACCTAAAACTACCAGTCAGGACGGCCAAAACATA | 142 |
| GCAGACGAAAGAGAAAAATCTACGTTCAACTAAGACG | 143 |
| ATGGAATACATTAGCCGCTGACCTTCATCAAACACTATGGAATTA | 144 |
| CACAAACGGGCCTAAAACGGTCAACAGACCAGGCGCATACCAGACTGCAGAT | 145 |
| TTAGGCTTTGTTTGACCCCGCGACCTTGACAAGAACCGCTTG | 146 |
| AGGACATCCACGAGCTGTAATGCTTAAGAGGTGAC | 147 |
| AGATTAAGCAAAATGGTCGGTGTCAACAGGTGTCA | 148 |
| TTTACCGTAACAACTAAAAATCTCGGGATCGGAGG | 149 |
| CCGTGTAGCAAACAACTTCGGTTTTTCGGTCTTTC | 150 |
| GTAAGGTTTACTCATAGTTTCTGTGAGGTGACGCC | 151 |
| TCAGCCACCCACCAGTACGGAGTGCAAAAGGGGAG | 152 |
| AACAAATCAGACGATTGGCCT | 153 |
| GCCAGAATGACAGGGCGTTTGTAATCAGATTAAAGAATT | 154 |
| TCTGAATCACCAGATCAAAATAAACCATTCAC | 155 |
| AGCGTCACAGAGCCCCACCACAGGCCGGGAATTAGAAGA | 156 |
| GCCTCCCAGAGCCACCACCCTTACATGGTTGAGTAATAA | 157 |
| GCATTTTCGGTCAAGTTTGCAAATATTCCAA | 158 |
| CTTATTAAGGTTGAGGCAGGTAAATCCTAAACATGAGGATTACTCA | 159 |
| TTCATAAGCCGCCGCCAGCATTGGAAAGATTTCGGTTGC | 160 |
| ACCAGAGGCCACCAGAACCACTTACCGTAACAGTTAGTGCCGAGAA | 161 |
| CGAATTTTGTCTGGCATGAGCAAGAGCGCATAGCC | 162 |
| AGAGGAAACCCCTTTTTAAAATAGTCCC | 163 |
| CATATAACGGCAATAGCAGAATAAAATAAACAACC | 164 |
| CACATTACGCTTGAGTTAACTGAACGCTAACTGAA | 165 |
| TAACTCCCGATTATCAACATTTTCGAGCCAGTGCG | 166 |
| AATTGAACAAAACATGTAATTTAGAGTATAAGTACCGC | 167 |
| GCCGCTAATGAGAATATAAAGTACAAAA | 168 |
| GCCTCGTAGGAATCATTATAGCAAAAGA | 169 |
| TTAAGCAAGCCGTTTTTGAAGGCTGAGG | 170 |
| ATATAATAAGCAGAACGCGCCTGTCTTGCGGTATCCGG | 171 |
| TACTACTAGACGACAAAAAACAACATGTTCATTAAATCGCAAATC | 172 |
| TTATCTTACCGCAGAGGCAATAGATAAGTCCCAGTTTTAGCG | 173 |
| GAAGAATTACTATGTGATGTAAATGCGAGAAGCTC | 174 |
| ACGACATCAAATTAATTTACCTTTCTGACCTTATC | 175 |
| TACCTTTGAACAAAAGATTGAAAAAAAATCAGAAA | 176 |
| TTAACCTTTTTTACATTTTCTGTAAGGTTGGTATT | 177 |
| CTCAAATAATCTAACAGACAACAGAGATCTTG | 178 |
| CAATATCGAGAGCCTATTAGTTAAAAGGAAACTATAAAA | 179 |
| ATCAACACGCCTGCCTGATAGCACCAGTATAT | 180 |
| AGGAAGGTGAGGCGCATTAAAGATTATTCCAGCCAGAAT | 181 |
| AACGAACAGATAAAACAGAGGTTATCTATACATTTAGATGATATTA | 182 |
| GAATGGCAGCAGCAAATGAAAATCAAACTGCCCGAATCATTTAGAAATAAGAT | 183 |
| GCGCGAAAACAGTGCCACGCTTGGTCAGGACAACTACCACCAAAAA | 184 |
| ACATCGCGTCAGTATTAACACGTTGAAAATTAGACATCATCAAGGGTTAAATA | 185 |
| CCTAAATTAAACTATGGTAACCACCCAC | 186 |
| CCATAATCAGTTAGAATCGGGCGCAGGT | 187 |
| CCTAACAGGAAAGGGAACGGAACCTAGC | 188 |
| GAGACGTATAGTGTAGCAAATCAAAGTC | 189 |
| TACGACTCCACAACATAATAGCTGCCCTGCATCAG | 190 |
| GCCGTTGTTCCAGCAGGTGAGGATGCACTCTGTG | 191 |
| CCGGATGGTGGTCCGTGAGAATGCGGCGGGCCCCTGCGTATTGGG | 192 |
| CACAAAGTGTCTCGAATGCAGTGTCA | 193 |
| GACATCAAAAGGGGGTAAAAACCACGGTGTATCAT | 194 |
| TTAGTCTTTATGCGGAAAAGAGCAGAGTAATCTG | 195 |
| TGCCTGTTCTTCGCGTTCCGATTGCCCT | 196 |
| ACGTTCAGCAGGGAAACCTGTCGTTGCCTAAGAAG | 197 |
| CTGCGAACATCCGCATTAATGAATCGCAAGCGGTCCACAGCCTG | 198 |
| GTGCAAGGTTTCGCGGGGAGAGGCGCTGGCCCCCTG | 199 |
| GCATGGTGTGATCCAGCTCGTAATCATGGTCCGAGCCGTGAGTGA | 200 |
| ATTATGGGTATGCGGCCAGCCTCCTCACAGTCGAAAATTGAGAGA | 201 |
| TAAGGAGGTGCGTCATACGCCTGTCCCCGGGTACCGAGAAGCTGTTTG | 202 |
| GGCCGGCCAGACAGCGGAAAAAAGCTTGTAGCCAC | 203 |
| GGAGGATAACCCGTGGTTAAACGAAGCCTCCGGTC | 204 |
| TTTCTGCAAGAGTCACGTCTGCTCTCGACATGCTG | 205 |
| TCCGGGCCTCAGGTGGACACGGAATTTAGTGCTCA | 206 |
| CATCATTGAAAATAAGGGATATTCAATTGTGTCGA | 207 |
| GAAAGACTGGCTCGTTTAGGCTGGGAACGAGGC | 208 |
| ATGTGAGCCAATAGGAACGCCATGAACGTTTT | 209 |
| GATAGGTTGTAAACGTTAATACCCAAAAAATCACCGTGT | 210 |
| ACGGCGGAAAATTCGCATTAACCGGTTGCAAC | 211 |
| TCGGATTTTAAATCAGCTCATAGCATGTTAATGCCTTTT | 212 |

1. A method of calibrating a microfluidic system comprising:
a. providing at least one bead having a structure based on DNA origami, wherein said structure comprises a predetermined number of fluorophores;
b. measuring the fluorescence of said at least one bead; and
c. calibrating the microfluidic system on the basis of the fluorescence measurement of b.
thereby obtaining a calibration curve of the microfluidic system.

2. The method according to claim 1, wherein said microfluidic system is a flow cytometer

3. The method according to any one of the preceding claims, wherein the structure comprises a predetermined number of fluorophores, a single stranded DNA molecule and staple DNA strands, wherein the number of staple DNA strands (Ns) is as in formula I:
Ns=Lss/n,
wherein Lss is the length of the single stranded DNA molecule, and
wherein n is an integer number comprised between 26 and 34.

4. The method according to any one of the preceding claims, wherein the single stranded DNA molecule has a length in a range between 1.500-nucleotides and 70.000-nucleotides, preferably between 2000-nucleotides and 68.000-nucleotides, such as between 5.000 and 65.000-nucleotides, for example between 10.000-nucleotides and 60.000-nucleotides, such as between 15.000-nucleotides and 55.000-nucleotides, preferably between 20.000-nucleotides and 55.000-nucleotides.

5. The method according to any one of the preceding claims, wherein the single stranded DNA molecule has a length of at least 2.000-nucleotides, such as at least 5.000-nucleotides, such as at least 7.000-nucleotides, such as at least 10.000-nucleotides, such as at least 15.000-nucleotides, such as at least 20.000-nucleotides, such as at least 25.000-nucleotides, such as at least 30.000-nucleotides, such as at least 35.000-nucleotides, such as at least 40.000-nucleotides, such as at least 45.000-nucleotides, such as at least 50.000-nucleotides.

6. The method according to any one of the preceding claims, wherein each staple DNA strand has a length in a range between 12 and 60 nucleotides.

7. The method according to any one of the preceding claims, wherein the structure comprises:
a. a positioning domain,
b. a fluorescent domain and/or
c. a triggering domain;

8. The method according to any one of the preceding claims, wherein the fluorescent domain comprises the predetermined number of fluorophores.

9. The method according to any one of the preceding claims, wherein the fluorophores are attached to the staple DNA strands.

10. The method according to any one of the preceding claims, wherein at least a part of the staple DNA strands comprises sticky ends, wherein the fluorophores are conjugated to a DNA fragment complementary to said sticky ends, and wherein the fluorophores are attached to said sticky ends via said complementary DNA fragment.

11. The method according to any one of the preceding claims, wherein the bead comprises a dimeric structure based on DNA origami.

12. The method according to any one of the preceding claims, wherein the bead comprises a trimeric structure based on DNA origami.

13. The method according to any one of the preceding claims, wherein the structure is a sphere, a barrel, a tube, a hexagon, or a combination thereof.

14. The method according to any one of the preceding claims, wherein the structure comprises at least 1 fluorophore, such as at least 50 fluorophores, such as at least 100 fluorophores, such as at least 200 fluorophores, such as at least 300 fluorophores, such as at least 400 fluorophores, such as at least 900 fluorophores, such as at least 1200 fluorophores, such as at least 1500 fluorophores, such as at least 1800 fluorophores, such as at least 2000 fluorophores, such as at least 2700 fluorophores.

15. The method according to any one of the preceding claims, wherein each bead comprises two or more different types of fluorophores or fluorescent domains.

16. The method according to any one of the preceding claims, wherein the fluorophore is selected from a group consisting of Quasar® 566 nm (Cy 3), Quasar® 670 nm (Cy 5), and fluorescein isothiocyanate (FITC).

17. The method according to any one of the preceding claims, wherein at least two beads, preferably at least three beads, are provided, and wherein each bead comprises a different predetermined number of fluorophores.

18. The method according to any one of the preceding claims, comprising measuring the fluorescence of the at least two, preferably at least three beads.

19. The method according to any one of the preceding claims, comprising calibrating the microfluidics system such as a flow cytometer on the basis of the fluorescence measurements of the at least two, preferably at least three beads.

20. A method for determining the level of an antigen in a sample by a microfluidics system comprising
a. calibrating a microfluidics system according to the method of any one of the preceding claims, thereby obtaining a calibration curve;
b. providing a sample comprising at least one antigen, wherein said antigen is attached to a cell or to a vesicle or to a virus-like particle or a viral particle or an extracellular vesicle;
c. contacting the sample with at least one ligand binding molecule coupled to a fluorescent label, wherein said ligand binds the at least one antigen;
d. measuring the fluorescence of said fluorescence label;
e. scoring the fluorescence of said fluorescence label based on the calibration curve, and
f. determining the level of said at least one antigen in the sample.

21. The method according to claim 20, wherein the level of an antigen is determined by flow cytometry.

22. The method according to any one of claims 20 and 21, wherein the sample comprises at least one antigen, such as at least 10 antigens, for example at least 25 antigens, preferably at least 50 antigens, wherein said antigen or said antigens are attached to one or more cells and/or vesicles and/or virus-like particles and/or viral particles and/or exosomes.

23. A method for determining the presence or state of a haematological disease in an individual by a microfluidics system, the method comprising
a. calibrating a microfluidics system according to the method disclosed herein, thereby obtaining a calibration curve;
b. providing a fluid sample from said individual comprising at least one blood cell;
c. contacting the sample with at least one ligand binding molecule coupled to a fluorescent label, wherein said ligand is associated with the presence or state of said haematological disease;
d. measuring the fluorescence of said fluorescent label;
e. scoring the fluorescence of said fluorescent label based on the calibration curve; and
f. determining the level and/or concentration of said at least one antigen associated with the presence or state of said haematological disease.

24. The method of claim 23, wherein the fluid sample is selected from a group consisting of blood, bone marrow, spinal fluid, bronchoalveolar lavage and serous effusions.

25. The method of any one of claims 23 and 24, comprising the further step of comparing said level and/or concentration of said at least one antigen with a cut-off value,
wherein said cut-off value is determined from the concentration range of said at least one antigen in healthy individuals, such as individuals who do not present with a haematological disease, and
wherein a level and/or concentration that is lower or greater than the cut-off value indicates the presence or state of said haematological disease.

26. A kit for calibration of microfluidic instruments comprising a bead having a structure based on DNA origami wherein said structure comprises a predetermined number of fluorophores.

27. The kit of claim 26, wherein said bead is as defined in anyone of claims 2 to 14.

28. The kit according to any one of claims 26 and 27, wherein the kit comprises at least two beads, for example at least three beads, such as at least 4 beads, preferably at least 5 beads, wherein each bead comprises a different predetermined number of fluorophores.


SEQUENCE LISTING (212 sequences)

SEQ ID NO: 1 - DNA, 8064 bp, Artificial sequence
  DNA origami beads scaffold sequence
  misc_feature (1)..(8064): Scaffold sequence
ggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctca

SEQ ID NO: 2 - DNA, 28 bp, Artificial Sequence
  Staple strand sequence
  misc_feature (1)..(28): Staple strand sequence
atacaggaatgcctgagtaatatcaata

SEQ ID NO: 3 - DNA, 28 bp, Artificial Sequence
  Staple strand sequence
  misc_feature (1)..(28): Staple strand sequence
ctcagaggcaaggataaaaatggagagg

SEQ ID NO: 4 - DNA, 28 bp, Artificial Sequence
  Staple strand sequence
  misc_feature (1)..(28): Staple strand sequence
ttcccaagagtagatttagttctaaatc

SEQ ID NO: 5 - DNA, 28 bp, Artificial Sequence
  Staple strand sequence
  misc_feature (1)..(28): Staple strand sequence
ctgaataaaaaggtggcatcatagtagt

SEQ ID NO: 6 - DNA, 28 bp, Artificial Sequence
  Staple strand sequence
  misc_feature (1)..(28): Staple strand sequence
taaagtacaataacctgtttaaagaatt

SEQ ID NO: 7 - DNA, 28 bp, Artificial Sequence
  Staple strand sequence
  misc_feature (1)..(28): Staple strand sequence
aaatcagattgctccttttgagtagctc

SEQ ID NO: 8 - DNA, 28 bp, Artificial Sequence
  Staple strand sequence
  misc_feature (1)..(28): Staple strand sequence
ggattgccggaagcaaactcctggaagt

SEQ ID NO: 9 - DNA, 24 bp, Artificial Sequence
  Staple strand sequence
  misc_feature (1)..(24): Staple strand sequence
ggcgaagttttgccagaagattaa

SEQ ID NO: 10 - DNA, 24 bp, Artificial Sequence
  Staple strand sequence
  misc_feature (1)..(24): Staple strand sequence
gtgtccccctcaaatgctcagaaa

SEQ ID NO: 11 - DNA, 24 bp, Artificial Sequence
  Staple strand sequence
  misc_feature (1)..(24): Staple strand sequence
accatagcgtccaatacccctgac

SEQ ID NO: 12 - DNA, 28 bp, Artificial Sequence
  Staple strand sequence
  misc_feature (1)..(28): Staple strand sequence
ggttgtagggagaagcctttagagatct

SEQ ID NO: 13 - DNA, 28 bp, Artificial Sequence
  Staple strand sequence
  misc_feature (1)..(28): Staple strand sequence
agcattataaagattcaaaaggccggag

SEQ ID NO: 14 - DNA, 28 bp, Artificial Sequence
  Staple strand sequence
  misc_feature (1)..(28): Staple strand sequence
agcaaaaaccctcatatattttctagct

SEQ ID NO: 15 - DNA, 28 bp, Artificial Sequence
  Staple strand sequence
  misc_feature (1)..(28): Staple strand sequence
aacatgttttcatttggggcgataaatc

SEQ ID NO: 16 - DNA, 28 bp, Artificial Sequence
  Staple strand sequence
  misc_feature (1)..(28): Staple strand sequence
ttcattctagatacatttcgcataaagc

SEQ ID NO: 17 - DNA, 28 bp, Artificial Sequence
  Staple strand sequence
  misc_feature (1)..(28): Staple strand sequence
gaggaaggagcttcaaagcgacagttga

SEQ ID NO: 18 - DNA, 28 bp, Artificial Sequence
  Staple strand sequence
  misc_feature (1)..(28): Staple strand sequence
acgagaatcatttttgcggatttaattg

SEQ ID NO: 19 - DNA, 28 bp, Artificial Sequence
  Staple strand sequence
  misc_feature (1)..(28): Staple strand sequence
tattatacaggattagagagtatgcaac

SEQ ID NO: 20 - DNA, 24 bp, Artificial Sequence
  Staple strand sequence
  misc_feature (1)..(24): Staple strand sequence
ataatagtaaaatgtttgcaaagc

SEQ ID NO: 21 - DNA, 31 bp, Artificial Sequence
  Staple strand sequence
  misc_feature (1)..(31): Staple strand sequence
ccctgacggttcgtcataaatattaaatcaa

SEQ ID NO: 22 - DNA, 42 bp, Artificial Sequence
  Staple strand sequence
  misc_feature (1)..(42): Staple strand sequence
acataacttgggaaggcaaaaaggaagtttccattgcataac

SEQ ID NO: 23 - DNA, 31 bp, Artificial Sequence
  Staple strand sequence
  misc_feature (1)..(31): Staple strand sequence
gcgattatacgcaacggctacagaaaggccg

SEQ ID NO: 24 - DNA, 28 bp, Artificial Sequence
  Staple strand sequence
  misc_feature (1)..(28): Staple strand sequence
caaccatatttcttaaacagcgttagta

SEQ ID NO: 25 - DNA, 28 bp, Artificial Sequence
  Staple strand sequence
  misc_feature (1)..(28): Staple strand sequence
cagcagcttttttcacgttgaaggaatt

SEQ ID NO: 26 - DNA, 28 bp, Artificial Sequence
  Staple strand sequence
  misc_feature (1)..(28): Staple strand sequence
cttgcagagcctttaattgtattcaaca

SEQ ID NO: 27 - DNA, 28 bp, Artificial Sequence
  Staple strand sequence
  misc_feature (1)..(28): Staple strand sequence
aaaggaacactgagtttcgtctcagagc

SEQ ID NO: 28 - DNA, 28 bp, Artificial Sequence
  Staple strand sequence
  misc_feature (1)..(28): Staple strand sequence
tttgctattccacagacagccgtaccgc

SEQ ID NO: 29 - DNA, 42 bp, Artificial Sequence
  Staple strand sequence
  misc_feature (1)..(42): Staple strand sequence
cgaggcactcattaactcatcaggactaaagacttgctgagg

SEQ ID NO: 30 - DNA, 24 bp, Artificial Sequence
  Staple strand sequence
  misc_feature (1)..(24): Staple strand sequence
caataaaatacgtaatgatgacaa

SEQ ID NO: 31 - DNA, 28 bp, Artificial Sequence
  Staple strand sequence
  misc_feature (1)..(28): Staple strand sequence
agtacaagacagcatcggaactcaccct

SEQ ID NO: 32 - DNA, 28 bp, Artificial Sequence
  Staple strand sequence
  misc_feature (1)..(28): Staple strand sequence
cttttgccaaaaaaaaggctcagaatag

SEQ ID NO: 33 - DNA, 28 bp, Artificial Sequence
  Staple strand sequence
  misc_feature (1)..(28): Staple strand sequence
cgatataatcagcttgctttcatgggat

SEQ ID NO: 34 - DNA, 28 bp, Artificial Sequence
  Staple strand sequence
  misc_feature (1)..(28): Staple strand sequence
aatgaatttagcgtaacgatctcaccgt

SEQ ID NO: 35 - DNA, 28 bp, Artificial Sequence
  Staple strand sequence
  misc_feature (1)..(28): Staple strand sequence
gcgaataataggaacccatgttcaggga

SEQ ID NO: 36 - DNA, 28 bp, Artificial Sequence
  Staple strand sequence
  misc_feature (1)..(28): Staple strand sequence
gtttcagcaaactacaacgccccaccct

SEQ ID NO: 37 - DNA, 28 bp, Artificial Sequence
  Staple strand sequence
  misc_feature (1)..(28): Staple strand sequence
attattctagcgacagaatcatagcccc

SEQ ID NO: 38 - DNA, 28 bp, Artificial Sequence
  Staple strand sequence
  misc_feature (1)..(28): Staple strand sequence
catttggaaacgtcaccaatgcaccgga

SEQ ID NO: 39 - DNA, 28 bp, Artificial Sequence
  Staple strand sequence
  misc_feature (1)..(28): Staple strand sequence
tagcaaaataaaagaaacgcaagccagc

SEQ ID NO: 40 - DNA, 28 bp, Artificial Sequence
  Staple strand sequence
  misc_feature (1)..(28): Staple strand sequence
accagaacaaaagggcgacatgagggag

SEQ ID NO: 41 - DNA, 28 bp, Artificial Sequence
  Staple strand sequence
  misc_feature (1)..(28): Staple strand sequence
aaaagaacacaatcaatagaagtgaatt

SEQ ID NO: 42 - DNA, 28 bp, Artificial Sequence
  Staple strand sequence
  misc_feature (1)..(28): Staple strand sequence
tacagagtatcttaccgaagcgaggaaa

SEQ ID NO: 43 - DNA, 28 bp, Artificial Sequence
  Staple strand sequence
  misc_feature (1)..(28): Staple strand sequence
gagaattaagcccaataataagattaag

SEQ ID NO: 44 - DNA, 24 bp, Artificial Sequence
  Staple strand sequence
  misc_feature (1)..(24): Staple strand sequence
aatccaaataagaaacgtaacgtc

SEQ ID NO: 45 - DNA, 24 bp, Artificial Sequence
  Staple strand sequence
  misc_feature (1)..(24): Staple strand sequence
ttactgaatcttaccaacaccctg

SEQ ID NO: 46 - DNA, 24 bp, Artificial Sequence
  Staple strand sequence
  misc_feature (1)..(24): Staple strand sequence
aactttgccagttacaacataaaa

SEQ ID NO: 47 - DNA, 28 bp, Artificial Sequence
  Staple strand sequence
  misc_feature (1)..(28): Staple strand sequence
aaaatcacattaccattagcacggaacc

SEQ ID NO: 48 - DNA, 29 bp, Artificial Sequence
  Staple strand sequence
  misc_feature (1)..(29): Staple strand sequence
ggaaggtctttagcgtcagactttcatcg

SEQ ID NO: 49 - DNA, 28 bp, Artificial Sequence
  Staple strand sequence
  misc_feature (1)..(28): Staple strand sequence
atcaccgcgatagcagcaccgccatctt

SEQ ID NO: 50 - DNA, 28 bp, Artificial Sequence
  Staple strand sequence
  misc_feature (1)..(28): Staple strand sequence
cgcaataatggtttaccagcggacggaa

SEQ ID NO: 51 - DNA, 28 bp, Artificial Sequence
  Staple strand sequence
  misc_feature (1)..(28): Staple strand sequence
actccttcacggaataagtttcttgagc

SEQ ID NO: 52 - DNA, 28 bp, Artificial Sequence
  Staple strand sequence
  misc_feature (1)..(28): Staple strand sequence
aacaaagataacccacaagaaagtatgt

SEQ ID NO: 53 - DNA, 28 bp, Artificial Sequence
  Staple strand sequence
  misc_feature (1)..(28): Staple strand sequence
aaaaatgaagaaaagtaagcacaaagtt

SEQ ID NO: 54 - DNA, 28 bp, Artificial Sequence
  Staple strand sequence
  misc_feature (1)..(28): Staple strand sequence
acagggaaaacaatgaaatagaataccc

SEQ ID NO: 55 - DNA, 24 bp, Artificial Sequence
  Staple strand sequence
  misc_feature (1)..(24): Staple strand sequence
agaagccatattatttacagcctt

SEQ ID NO: 56 - DNA, 24 bp, Artificial Sequence
  Staple strand sequence
  misc_feature (1)..(24): Staple strand sequence
tttgagcgtctttccagtagacgg

SEQ ID NO: 57 - DNA, 42 bp, Artificial Sequence
  Staple strand sequence
  misc_feature (1)..(42): Staple strand sequence
agatataattttcatgtttagaaatttaatggttttaggtct

SEQ ID NO: 58 - DNA, 39 bp, Artificial Sequence
  Staple strand sequence
  misc_feature (1)..(39): Staple strand sequence
actcatcagaacgcgaggcataatcggcaaaccaaagcc

SEQ ID NO: 59 - DNA, 24 bp, Artificial Sequence
  Staple strand sequence
  misc_feature (1)..(24): Staple strand sequence
aacaactttttcaaatagttatat

SEQ ID NO: 60 - DNA, 28 bp, Artificial Sequence
  Staple strand sequence
  misc_feature (1)..(28): Staple strand sequence
atttatccatagcgatagcttttcaatt

SEQ ID NO: 61 - DNA, 28 bp, Artificial Sequence
  Staple strand sequence
  misc_feature (1)..(28): Staple strand sequence
caaatcctacataaatcaatactttttt

SEQ ID NO: 62 - DNA, 28 bp, Artificial Sequence
  Staple strand sequence
  misc_feature (1)..(28): Staple strand sequence
ccggcttaatcgtcgctattagaaaaca

SEQ ID NO: 63 - DNA, 28 bp, Artificial Sequence
  Staple strand sequence
  misc_feature (1)..(28): Staple strand sequence
ttcattttatacagtaacagttttgcac

SEQ ID NO: 64 - DNA, 28 bp, Artificial Sequence
  Staple strand sequence
  misc_feature (1)..(28): Staple strand sequence
gaaacaagattcgcctgattgttctgaa

SEQ ID NO: 65 - DNA, 28 bp, Artificial Sequence
  Staple strand sequence
  misc_feature (1)..(28): Staple strand sequence
aatggaatcaggtttaacgtcaagaaat

SEQ ID NO: 66 - DNA, 28 bp, Artificial Sequence
  Staple strand sequence
  misc_feature (1)..(28): Staple strand sequence
gagagacttcccttagaatccagatgat

SEQ ID NO: 67 - DNA, 42 bp, Artificial Sequence
  Staple strand sequence
  misc_feature (1)..(42): Staple strand sequence
tattctagagaacatacaaatgttaatttcatcttttaacct

SEQ ID NO: 68 - DNA, 28 bp, Artificial Sequence
  Staple strand sequence
  misc_feature (1)..(28): Staple strand sequence
aaattaaacatcgggagaaacgaaccta

SEQ ID NO: 69 - DNA, 28 bp, Artificial Sequence
  Staple strand sequence
  misc_feature (1)..(28): Staple strand sequence
cttaattgcaagacaaagaacgctgatg

SEQ ID NO: 70 - DNA, 28 bp, Artificial Sequence
  Staple strand sequence
  misc_feature (1)..(28): Staple strand sequence
aactatagtgaataaccttgctaacaat

SEQ ID NO: 71 - DNA, 35 bp, Artificial Sequence
  Staple strand sequence
  misc_feature (1)..(35): Staple strand sequence
accggaatcataatcgaccgtgtgataaatagtga

SEQ ID NO: 72 - DNA, 28 bp, Artificial Sequence
  Staple strand sequence
  misc_feature (1)..(28): Staple strand sequence
acctgagtaccaagttacaaatcctgat

SEQ ID NO: 73 - DNA, 28 bp, Artificial Sequence
  Staple strand sequence
  misc_feature (1)..(28): Staple strand sequence
agaactcgacattctggccaatattttt

SEQ ID NO: 74 - DNA, 28 bp, Artificial Sequence
  Staple strand sequence
  misc_feature (1)..(28): Staple strand sequence
attaccgtacattggcagattccctaaa

SEQ ID NO: 75 - DNA, 28 bp, Artificial Sequence
  Staple strand sequence
  misc_feature (1)..(28): Staple strand sequence
taaagggggaacggtacgccattgcaac

SEQ ID NO: 76 - DNA, 28 bp, Artificial Sequence
  Staple strand sequence
  misc_feature (1)..(28): Staple strand sequence
ggcgcgtccgttgtagcaatatagtaat

SEQ ID NO: 77 - DNA, 28 bp, Artificial Sequence
  Staple strand sequence
  misc_feature (1)..(28): Staple strand sequence
ttcctcgtgaggccaccgagtcggcctt

SEQ ID NO: 78 - DNA, 28 bp, Artificial Sequence
  Staple strand sequence
  misc_feature (1)..(28): Staple strand sequence
atcacccggtcacgctgcgcgttgcttt

SEQ ID NO: 79 - DNA, 28 bp, Artificial Sequence
  Staple strand sequence
  misc_feature (1)..(28): Staple strand sequence
actaaatgaaagcgaaaggagcagagcg

SEQ ID NO: 80 - DNA, 28 bp, Artificial Sequence
  Staple strand sequence
aatcggcataaatcaaaagaactaaagg

SEQ ID NO: 81 - DNA, 24 bp, Artificial Sequence
  Staple strand sequence
  misc_feature (1)..(24): Staple strand sequence
acaacgtcaaagggcgaatcaggg

SEQ ID NO: 82 - DNA, 24 bp, Artificial Sequence
  Staple strand sequence
  misc_feature (1)..(24): Staple strand sequence
ccccagtttggaacaaggtttttt

SEQ ID NO: 83 - DNA, 28 bp, Artificial Sequence
  Staple strand sequence
  misc_feature (1)..(28): Staple strand sequence
aggaaaaaatcgtctgaaatgaataccg

SEQ ID NO: 84 - DNA, 28 bp, Artificial Sequence
  Staple strand sequence
  misc_feature (1)..(28): Staple strand sequence
aacatcaagaacccttctgacaagaata

SEQ ID NO: 85 - DNA, 28 bp, Artificial Sequence
  Staple strand sequence
  misc_feature (1)..(28): Staple strand sequence
gctggtacacacgaccagtaactttaat

SEQ ID NO: 86 - DNA, 28 bp, Artificial Sequence
  Staple strand sequence
  misc_feature (1)..(28): Staple strand sequence
gacgagctctgtccatcacgcgagtaga

SEQ ID NO: 87 - DNA, 28 bp, Artificial Sequence
  Staple strand sequence
  misc_feature (1)..(28): Staple strand sequence
ggagctagagaagtgtttttagaacaat

SEQ ID NO: 88 - DNA, 28 bp, Artificial Sequence
  Staple strand sequence
  misc_feature (1)..(28): Staple strand sequence
gagcccccgtggcgagaaaggggccgat

SEQ ID NO: 89 - DNA, 28 bp, Artificial Sequence
  Staple strand sequence
  misc_feature (1)..(28): Staple strand sequence
cgatggccacacccgccgcgcgctacag

SEQ ID NO: 90 - DNA, 28 bp, Artificial Sequence
  Staple strand sequence
  misc_feature (1)..(28): Staple strand sequence
ggggtcgtagggcgctggcaaacgtgct

SEQ ID NO: 91 - DNA, 24 bp, Artificial Sequence
  Staple strand sequence
  misc_feature (1)..(24): Staple strand sequence
cattattaaagaacgtggtgaacc

SEQ ID NO: 92 - DNA, 24 bp, Artificial Sequence
  Staple strand sequence
  misc_feature (1)..(24): Staple strand sequence
tttagatagggttgagtgtaaagc

SEQ ID NO: 93 - DNA, 42 bp, Artificial Sequence
  Staple strand sequence
  misc_feature (1)..(42): Staple strand sequence
gctaacttccagtcaatcgtttcggtggtgccatcaacgtca

SEQ ID NO: 94 - DNA, 28 bp, Artificial Sequence
  Staple strand sequence
  misc_feature (1)..(28): Staple strand sequence
tagctctgccgccacgggaacagggcga

SEQ ID NO: 95 - DNA, 28 bp, Artificial Sequence
  Staple strand sequence
  misc_feature (1)..(28): Staple strand sequence
tgccggaccgcacaggcggccaaagaga

SEQ ID NO: 96 - DNA, 28 bp, Artificial Sequence
  Staple strand sequence
  misc_feature (1)..(28): Staple strand sequence
cgctggctgctgattgccgttgagatag

SEQ ID NO: 97 - DNA, 28 bp, Artificial Sequence
  Staple strand sequence
  misc_feature (1)..(28): Staple strand sequence
ttaaattacgttgtaaaacgagaaaggg

SEQ ID NO: 98 - DNA, 28 bp, Artificial Sequence
  Staple strand sequence
  misc_feature (1)..(28): Staple strand sequence
cagcaacggcggttgtgtacaatttgcc

SEQ ID NO: 99 - DNA, 42 bp, Artificial Sequence
  Staple strand sequence
  misc_feature (1)..(42): Staple strand sequence
gttgcaggccaacgctttgcttccagcatcagcggggccaga

SEQ ID NO: 100 - DNA, 42 bp, Artificial Sequence
  Staple strand sequence
  misc_feature (1)..(42): Staple strand sequence
tcaccgcgtttgcggctggtagcaggcgctttcgcgtctcgt

SEQ ID NO: 101 - DNA, 38 bp, Artificial Sequence
  Staple strand sequence
  misc_feature (1)..(38): Staple strand sequence
ggggccagctcttacacaccagcttacggctcggaacg

SEQ ID NO: 102 - DNA, 28 bp, Artificial Sequence
  Staple strand sequence
  misc_feature (1)..(28): Staple strand sequence
ccgggttagaatgccaacggctggtcag

SEQ ID NO: 103 - DNA, 28 bp, Artificial Sequence
  Staple strand sequence
  misc_feature (1)..(28): Staple strand sequence
gcgtggtaaaaaaatcccgtaatcaaac

SEQ ID NO: 104 - DNA, 28 bp, Artificial Sequence
  Staple strand sequence
  misc_feature (1)..(28): Staple strand sequence
gcacatcatgaagggtaaagtgaaggga

SEQ ID NO: 105 - DNA, 28 bp, Artificial Sequence
  Staple strand sequence
  misc_feature (1)..(28): Staple strand sequence
actttctctcaccggaaacaacaggctg

SEQ ID NO: 106 - DNA, 28 bp, Artificial Sequence
  Staple strand sequence
  misc_feature (1)..(28): Staple strand sequence
gccagcagccagggttttcccgcgatta

SEQ ID NO: 107 - DNA, 28 bp, Artificial Sequence
  Staple strand sequence
  misc_feature (1)..(28): Staple strand sequence
cgcagaatgccaagctttcagttcgcta

SEQ ID NO: 108 - DNA, 43 bp, Artificial Sequence
  Staple strand sequence
  misc_feature (1)..(43): Staple strand sequence
actcaggtagcccggaataggacggggtcagtgcccttttgat

SEQ ID NO: 109 - DNA, 42 bp, Artificial Sequence
  Staple strand sequence
  misc_feature (1)..(42): Staple strand sequence
tagcaaggactcctcaagagaaaagtattaagaggattcaca

SEQ ID NO: 110 - DNA, 42 bp, Artificial Sequence
  Staple strand sequence
  misc_feature (1)..(42): Staple strand sequence
caccaccggattagcggggttaacctattattctgcattaaa

SEQ ID NO: 111 - DNA, 42 bp, Artificial Sequence
  Staple strand sequence
  misc_feature (1)..(42): Staple strand sequence
cagaaccgtaccaggcggataaatgccccctgcctcgcagtc

SEQ ID NO: 112 - DNA, 42 bp, Artificial Sequence
  Staple strand sequence
  misc_feature (1)..(42): Staple strand sequence
caccctctcgagagggttgatacagtgcccgtatatccagta

SEQ ID NO: 113 - DNA, 42 bp, Artificial Sequence
  Staple strand sequence
  misc_feature (1)..(42): Staple strand sequence
tgtttggggcaattcatcaatccgtcaatagataaaaatatc

SEQ ID NO: 114 - DNA, 42 bp, Artificial Sequence
  Staple strand sequence
  misc_feature (1)..(42): Staple strand sequence
tgcgtaggtttgagtaacattacgttattaattttgctgaac

SEQ ID NO: 115 - DNA, 42 bp, Artificial Sequence
  Staple strand sequence
  misc_feature (1)..(42): Staple strand sequence
gtaaaactgcggaacaaagaacgtattaaatccttcctcaat

SEQ ID NO: 116 - DNA, 42 bp, Artificial Sequence
  Staple strand sequence
  misc_feature (1)..(42): Staple strand sequence
ccatatcgaaggagcggaatttttacaaacaattcttggcaa

SEQ ID NO: 117 - DNA, 42 bp, Artificial Sequence
  Staple strand sequence
  misc_feature (1)..(42): Staple strand sequence
taatggatattcctgattatcgaggatttagaagtggaattg

SEQ ID NO: 118 - DNA, 28 bp, Artificial Sequence
  Staple strand sequence
  misc_feature (1)..(28): Staple strand sequence
ggatgtggaggggacgacgacgtaatgg

SEQ ID NO: 119 - DNA, 28 bp, Artificial Sequence
  Staple strand sequence
  misc_feature (1)..(28): Staple strand sequence
ttacgccgcctcaggaagatcggaacaa

SEQ ID NO: 120 - DNA, 28 bp, Artificial Sequence
  Staple strand sequence
  misc_feature (1)..(28): Staple strand sequence
tcggtgcagccagctttccggcaacccg

SEQ ID NO: 121 - DNA, 28 bp, Artificial Sequence
  Staple strand sequence
  misc_feature (1)..(28): Staple strand sequence
agttgggtaaccgtgcatctggtgtaga

SEQ ID NO: 122 - DNA, 28 bp, Artificial Sequence
  Staple strand sequence
  misc_feature (1)..(28): Staple strand sequence
cgcaacttctggtgccggaaaacattaa

SEQ ID NO: 123 - DNA, 35 bp, Artificial Sequence
  Staple strand sequence
  misc_feature (1)..(35): Staple strand sequence
caactttaatcattgtgaattaccgtaaattttca

SEQ ID NO: 124 - DNA, 22 bp, Artificial Sequence
  Staple strand sequence
  misc_feature (1)..(22): Staple strand sequence
agatttgtatcatcggaaacaa

SEQ ID NO: 125 - DNA, 36 bp, Artificial Sequence
  Staple strand sequence
  misc_feature (1)..(36): Staple strand sequence
gtacaagcgccctgataattacccaaatcaacgtaa

SEQ ID NO: 126 - DNA, 43 bp, Artificial Sequence
  Staple strand sequence
  misc_feature (1)..(43): Staple strand sequence
gccatatttaaagtagggcaagaacgggtatttgtctttatgt

SEQ ID NO: 127 - DNA, 39 bp, Artificial Sequence
  Staple strand sequence
  misc_feature (1)..(39): Staple strand sequence
aaccaacgccgaaaaataatatcctacgagcccttatca

SEQ ID NO: 128 - DNA, 21 bp, Artificial Sequence
  Staple strand sequence
  misc_feature (1)..(21): Staple strand sequence
atccgctcacaaattgcgttg

SEQ ID NO: 129 - DNA, 48 bp, Artificial Sequence
  Staple strand sequence
  misc_feature (1)..(48): Staple strand sequence
ctgcagccagcggtgctcagatgctcactgcccgcttcacattattcc

SEQ ID NO: 130 - DNA, 43 bp, Artificial Sequence
  Staple strand sequence
  misc_feature (1)..(43): Staple strand sequence
gtcagcaccgaacggcacggtgcctttcctgtgtgaaattgtt

SEQ ID NO: 131 - DNA, 29 bp, Artificial Sequence
  Staple strand sequence
  misc_feature (1)..(29): Staple strand sequence
caaagctgctcagggcttgagatggttta

SEQ ID NO: 132 - DNA, 35 bp, Artificial Sequence
  Staple strand sequence
  misc_feature (1)..(35): Staple strand sequence
tgatttcaaccataaagtgaccatcatataaacca

SEQ ID NO: 133 - DNA, 21 bp, Artificial Sequence
  Staple strand sequence
  misc_feature (1)..(21): Staple strand sequence
acaaaggaaacaagagaatcg

SEQ ID NO: 134 - DNA, 39 bp, Artificial Sequence
  Staple strand sequence
  misc_feature (1)..(39): Staple strand sequence
acagtcaacaggaagattgtatttaaatcacgttgccag

SEQ ID NO: 135 - DNA, 46 bp, Artificial Sequence
  Staple strand sequence
  misc_feature (1)..(46): Staple strand sequence
tgatattataatcagaaaagcttttgttattgaccagtatcgagct

SEQ ID NO: 136 - DNA, 35 bp, Artificial Sequence
  Staple strand sequence
  misc_feature (1)..(35): Staple strand sequence
cgttaaatgccaaggcagctatattttaaatacct

SEQ ID NO: 137 - DNA, 39 bp, Artificial Sequence
  Staple strand sequence
  misc_feature (1)..(39): Staple strand sequence
gataaatcaatcatatgtaccatttttgctccgtggcac

SEQ ID NO: 138 - DNA, 46 bp, Artificial Sequence
  Staple strand sequence
  misc_feature (1)..(46): Staple strand sequence
gtagctagtaatcgtaaaacttttttaacgagtaacaccgctgttg

SEQ ID NO: 139 - DNA, 39 bp, Artificial Sequence
  Staple strand sequence
  misc_feature (1)..(39): Staple strand sequence
agatgaaaaatagcaggaataccacattaataaaagcac

SEQ ID NO: 140 - DNA, 42 bp, Artificial Sequence
  Staple strand sequence
  misc_feature (1)..(42): Staple strand sequence
aagggaaccgtacgaagcgaactaacggaacgagatttgaga

SEQ ID NO: 141 - DNA, 49 bp, Artificial Sequence
  Staple strand sequence
  misc_feature (1)..(49): Staple strand sequence
aatcccagcgattttaagaactggtaagaaacaccagaacgagtattat

SEQ ID NO: 142 - DNA, 43 bp, Artificial Sequence
  Staple strand sequence
  misc_feature (1)..(43): Staple strand sequence
ctccatgttacctaaaactaccagtcaggacggccaaaacata

SEQ ID NO: 143 - DNA, 37 bp, Artificial Sequence
  Staple strand sequence
  misc_feature (1)..(37): Staple strand sequence
gcagacgaaagagaaaaatctacgttcaactaagacg

SEQ ID NO: 144 - DNA, 45 bp, Artificial Sequence
  Staple strand sequence
  misc_feature (1)..(45): Staple strand sequence
atggaatacattagccgctgaccttcatcaaacactatggaatta

SEQ ID NO: 145 - DNA, 52 bp, Artificial Sequence
  Staple strand sequence
  misc_feature (1)..(52): Staple strand sequence
cacaaacgggcctaaaacggtcaacagaccaggcgcataccagactgcagat

SEQ ID NO: 146 - DNA, 42 bp, Artificial Sequence
  Staple strand sequence
  misc_feature (1)..(42): Staple strand sequence
ttaggctttgtttgaccccgcgaccttgacaagaaccgcttg

SEQ ID NO: 147 - DNA, 35 bp, Artificial Sequence
  Staple strand sequence
  misc_feature (1)..(35): Staple strand sequence
aggacatccacgagctgtaatgcttaagaggtgac

SEQ ID NO: 148 - DNA, 35 bp, Artificial Sequence
  Staple strand sequence
  misc_feature (1)..(35): Staple strand sequence
agattaagcaaaatggtcggtgtcaacaggtgtca

SEQ ID NO: 149 - DNA, 35 bp, Artificial Sequence
  Staple strand sequence
  misc_feature (1)..(35): Staple strand sequence
tttaccgtaacaactaaaaatctcgggatcggagg

SEQ ID NO: 150 - DNA, 35 bp, Artificial Sequence
  Staple strand sequence
  misc_feature (1)..(35): Staple strand sequence
ccgtgtagcaaacaacttcggtttttcggtctttc

SEQ ID NO: 151 - DNA, 35 bp, Artificial Sequence
  Staple strand sequence
  misc_feature (1)..(35): Staple strand sequence
gtaaggtttactcatagtttctgtgaggtgacgcc

SEQ ID NO: 152 - DNA, 35 bp, Artificial Sequence
  Staple strand sequence
  misc_feature (1)..(35): Staple strand sequence
tcagccacccaccagtacggagtgcaaaaggggag

SEQ ID NO: 153 - DNA, 21 bp, Artificial Sequence
  Staple strand sequence
  misc_feature (1)..(21): Staple strand sequence
aacaaatcagacgattggcct

SEQ ID NO: 154 - DNA, 39 bp, Artificial Sequence
  Staple strand sequence
  misc_feature (1)..(39): Staple strand sequence
gccagaatgacagggcgtttgtaatcagattaaagaatt

SEQ ID NO: 155 - DNA, 32 bp, Artificial Sequence
  Staple strand sequence
  misc_feature (1)..(32): Staple strand sequence
tctgaatcaccagatcaaaataaaccattcac

SEQ ID NO: 156 - DNA, 39 bp, Artificial Sequence
  Staple strand sequence
  misc_feature (1)..(39): Staple strand sequence
agcgtcacagagccccaccacaggccgggaattagaaga

SEQ ID NO: 157 - DNA, 39 bp, Artificial Sequence
  Staple strand sequence
  misc_feature (1)..(39): Staple strand sequence
gcctcccagagccaccacccttacatggttgagtaataa

SEQ ID NO: 158 - DNA, 31 bp, Artificial Sequence
  Staple strand sequence
  misc_feature (1)..(31): Staple strand sequence
gcattttcggtcaagtttgcaaatattccaa

SEQ ID NO: 159 - DNA, 46 bp, Artificial Sequence
  Staple strand sequence
  misc_feature (1)..(46): Staple strand sequence
cttattaaggttgaggcaggtaaatcctaaacatgaggattactca

SEQ ID NO: 160 - DNA, 39 bp, Artificial Sequence
  Staple strand sequence
  misc_feature (1)..(39): Staple strand sequence
ttcataagccgccgccagcattggaaagatttcggttgc

SEQ ID NO: 161 - DNA, 46 bp, Artificial Sequence
  Staple strand sequence
  misc_feature (1)..(46): Staple strand sequence
accagaggccaccagaaccacttaccgtaacagttagtgccgagaa

SEQ ID NO: 162 - DNA, 35 bp, Artificial Sequence
  Staple strand sequence
  misc_feature (1)..(35): Staple strand sequence
cgaattttgtctggcatgagcaagagcgcatagcc

SEQ ID NO: 163 - DNA, 28 bp, Artificial Sequence
  Staple strand sequence
  misc_feature (1)..(28): Staple strand sequence
agaggaaacccctttttaaaatagtccc

SEQ ID NO: 164 - DNA, 35 bp, Artificial Sequence
  Staple strand sequence
  misc_feature (1)..(35): Staple strand sequence
catataacggcaatagcagaataaaataaacaacc

SEQ ID NO: 165 - DNA, 35 bp, Artificial Sequence
  Staple strand sequence
  misc_feature (1)..(35): Staple strand sequence
cacattacgcttgagttaactgaacgctaactgaa

SEQ ID NO: 166 - DNA, 35 bp, Artificial Sequence
  Staple strand sequence
  misc_feature (1)..(35): Staple strand sequence
taactcccgattatcaacattttcgagccagtgcg

SEQ ID NO: 167 - DNA, 38 bp, Artificial Sequence
  Staple strand sequence
  misc_feature (1)..(38): Staple strand sequence
aattgaacaaaacatgtaatttagagtataagtaccgc

SEQ ID NO: 168 - DNA, 28 bp, Artificial Sequence
  Staple strand sequence
  misc_feature (1)..(28): Staple strand sequence
gccgctaatgagaatataaagtacaaaa

SEQ ID NO: 169 - DNA, 28 bp, Artificial Sequence
  Staple strand sequence
  misc_feature (1)..(28): Staple strand sequence
gcctcgtaggaatcattatagcaaaaga

SEQ ID NO: 170 - DNA, 28 bp, Artificial Sequence
  Staple strand sequence
  misc_feature (1)..(28): Staple strand sequence
ttaagcaagccgtttttgaaggctgagg

SEQ ID NO: 171 - DNA, 38 bp, Artificial Sequence
  Staple strand sequence
  misc_feature (1)..(38): Staple strand sequence
atataataagcagaacgcgcctgtcttgcggtatccgg

SEQ ID NO: 172 - DNA, 45 bp, Artificial Sequence
  Staple strand sequence
  misc_feature (1)..(45): Staple strand sequence
tactactagacgacaaaaaacaacatgttcattaaatcgcaaatc

SEQ ID NO: 173 - DNA, 42 bp, Artificial Sequence
  Staple strand sequence
  misc_feature (1)..(42): Staple strand sequence
ttatcttaccgcagaggcaatagataagtcccagttttagcg

SEQ ID NO: 174 - DNA, 35 bp, Artificial Sequence
  Staple strand sequence
  misc_feature (1)..(35): Staple strand sequence
gaagaattactatgtgatgtaaatgcgagaagctc

SEQ ID NO: 175 - DNA, 35 bp, Artificial Sequence
  Staple strand sequence
  misc_feature (1)..(35): Staple strand sequence
acgacatcaaattaatttacctttctgaccttatc

SEQ ID NO: 176 - DNA, 35 bp, Artificial Sequence
  Staple strand sequence
  misc_feature (1)..(35): Staple strand sequence
tacctttgaacaaaagattgaaaaaaaatcagaaa

SEQ ID NO: 177 - DNA, 35 bp, Artificial Sequence
  Staple strand sequence
  misc_feature (1)..(35): Staple strand sequence
ttaaccttttttacattttctgtaaggttggtatt

SEQ ID NO: 178 - DNA, 32 bp, Artificial Sequence
  Staple strand sequence
  misc_feature (1)..(32): Staple strand sequence
ctcaaataatctaacagacaacagagatcttg

SEQ ID NO: 179 - DNA, 39 bp, Artificial Sequence
  Staple strand sequence
  misc_feature (1)..(39): Staple strand sequence
caatatcgagagcctattagttaaaaggaaactataaaa

SEQ ID NO: 180 - DNA, 32 bp, Artificial Sequence
  Staple strand sequence
  misc_feature (1)..(32): Staple strand sequence
atcaacacgcctgcctgatagcaccagtatat

SEQ ID NO: 181 - DNA, 39 bp, Artificial Sequence
  Staple strand sequence
  misc_feature (1)..(39): Staple strand sequence
aggaaggtgaggcgcattaaagattattccagccagaat

SEQ ID NO: 182 - DNA, 46 bp, Artificial Sequence
  Staple strand sequence
  misc_feature (1)..(46): Staple strand sequence
aacgaacagataaaacagaggttatctatacatttagatgatatta

SEQ ID NO: 183 - DNA, 53 bp, Artificial Sequence
  Staple strand sequence
  misc_feature (1)..(53): Staple strand sequence
gaatggcagcagcaaatgaaaatcaaactgcccgaatcatttagaaataagat

SEQ ID NO: 184 - DNA, 46 bp, Artificial Sequence
  Staple strand sequence
  misc_feature (1)..(46): Staple strand sequence
gcgcgaaaacagtgccacgcttggtcaggacaactaccaccaaaaa

SEQ ID NO: 185 - DNA, 53 bp, Artificial Sequence
  Staple strand sequence
  misc_feature (1)..(53): Staple strand sequence
acatcgcgtcagtattaacacgttgaaaattagacatcatcaagggttaaata

SEQ ID NO: 186 - DNA, 28 bp, Artificial Sequence
  Staple strand sequence
  misc_feature (1)..(28): Staple strand sequence
cctaaattaaactatggtaaccacccac

SEQ ID NO: 187 - DNA, 28 bp, Artificial Sequence
  Staple strand sequence
  misc_feature (1)..(28): Staple strand sequence
ccataatcagttagaatcgggcgcaggt

SEQ ID NO: 188 - DNA, 28 bp, Artificial Sequence
  Staple strand sequence
  misc_feature (1)..(28): Staple strand sequence
cctaacaggaaagggaacggaacctagc

SEQ ID NO: 189 - DNA, 28 bp, Artificial Sequence
  Staple strand sequence
  misc_feature (1)..(28): Staple strand sequence
gagacgtatagtgtagcaaatcaaagtc

SEQ ID NO: 190 - DNA, 35 bp, Artificial Sequence
  Staple strand sequence
  misc_feature (1)..(35): Staple strand sequence
tacgactccacaacataatagctgccctgcatcag

SEQ ID NO: 191 - DNA, 34 bp, Artificial Sequence
  Staple strand sequence
  misc_feature (1)..(34): Staple strand sequence
gccgttgttccagcaggtgaggatgcactctgtg

SEQ ID NO: 192 - DNA, 45 bp, Artificial Sequence
  Staple strand sequence
  misc_feature (1)..(45): Staple strand sequence
ccggatggtggtccgtgagaatgcggcgggcccctgcgtattggg

SEQ ID NO: 193 - DNA, 26 bp, Artificial Sequence
  Staple strand sequence
  misc_feature (1)..(26): Staple strand sequence
cacaaagtgtctcgaatgcagtgtca

SEQ ID NO: 194 - DNA, 35 bp, Artificial Sequence
  Staple strand sequence
  misc_feature (1)..(35): Staple strand sequence
gacatcaaaagggggtaaaaaccacggtgtatcat

SEQ ID NO: 195 - DNA, 34 bp, Artificial Sequence
  Staple strand sequence
  misc_feature (1)..(34): Staple strand sequence
ttagtctttatgcggaaaagagcagagtaatctg

SEQ ID NO: 196 - DNA, 28 bp, Artificial Sequence
  Staple strand sequence
  misc_feature (1)..(28): Staple strand sequence
tgcctgttcttcgcgttccgattgccct

SEQ ID NO: 197 - DNA, 35 bp, Artificial Sequence
  Staple strand sequence
  misc_feature (1)..(35): Staple strand sequence
acgttcagcagggaaacctgtcgttgcctaagaag

SEQ ID NO: 198 - DNA, 44 bp, Artificial Sequence
  Staple strand sequence
  misc_feature (1)..(44): Staple strand sequence
ctgcgaacatccgcattaatgaatcgcaagcggtccacagcctg

SEQ ID NO: 199 - DNA, 36 bp, Artificial Sequence
  Staple strand sequence
  misc_feature (1)..(36): Staple strand sequence
gtgcaaggtttcgcggggagaggcgctggccccctg

SEQ ID NO: 200 - DNA, 45 bp, Artificial Sequence
  Staple strand sequence
  misc_feature (1)..(45): Staple strand sequence
gcatggtgtgatccagctcgtaatcatggtccgagccgtgagtga

SEQ ID NO: 201 - DNA, 45 bp, Artificial Sequence
  Staple strand sequence
  misc_feature (1)..(45): Staple strand sequence
attatgggtatgcggccagcctcctcacagtcgaaaattgagaga

SEQ ID NO: 202 - DNA, 49 bp, Artificial Sequence
  Staple strand sequence
  misc_feature (1)..(49): Staple strand sequence
taaggaggtgcgtcatacgcctgtccccgggtaccgagaagctggtttg

SEQ ID NO: 203 - DNA, 35 bp, Artificial Sequence
  Staple strand sequence
  misc_feature (1)..(35): Staple strand sequence
ggccggccagacagcggaaaaaagcttgtagccac

SEQ ID NO: 204 - DNA, 35 bp, Artificial Sequence
  Staple strand sequence
  misc_feature (1)..(35): Staple strand sequence
ggaggataacccgtggttaaacgaagcctccggtc

SEQ ID NO: 205 - DNA, 35 bp, Artificial Sequence
  Staple strand sequence
  misc_feature (1)..(35): Staple strand sequence
tttctgcaagagtcacgtctgctctcgacatgctg

SEQ ID NO: 206 - DNA, 35 bp, Artificial Sequence
  Staple strand sequence
  misc_feature (1)..(35): Staple strand sequence
tccgggcctcaggtggacacggaatttagtgctca

SEQ ID NO: 207 - DNA, 35 bp, Artificial Sequence
  Staple strand sequence
  misc_feature (1)..(35): Staple strand sequence
catcattgaaaataagggatattcaattgtgtcga

SEQ ID NO: 208 - DNA, 33 bp, Artificial Sequence
  Staple strand sequence
  misc_feature (1)..(33): Staple strand sequence
gaaagactggctcgtttaggctgggaacgaggc

SEQ ID NO: 209 - DNA, 32 bp, Artificial Sequence
  Staple strand sequence
  misc_feature (1)..(32): Staple strand sequence
atgtgagccaataggaacgccatgaacgtttt

SEQ ID NO: 210 - DNA, 39 bp, Artificial Sequence
  Staple strand sequence
  misc_feature (1)..(39): Staple strand sequence
gataggttgtaaacgttaatacccaaaaaatcaccgtgt

SEQ ID NO: 211 - DNA, 32 bp, Artificial Sequence
  Staple strand sequence
  misc_feature (1)..(32): Staple strand sequence
acggcggaaaattcgcattaaccggttgcaac

SEQ ID NO: 212 - DNA, 39 bp, Artificial Sequence
  Staple strand sequence
  misc_feature (1)..(39): Staple strand sequence
tcggattttaaatcagctcatagcatgttaatgcctttt